(19) United States
(12) Patent Application Publication
(10) Pub. No.: US 2009/0291968 A1
(43) Pub. Date: Nov. 26, 2009

(54) SUBSTITUTED INDAZOLE DERIVATIVES, THEIR MANUFACTURE AND USE AS PHARMACEUTICAL AGENTS

(76) Inventors: Guy Georges, Habach (DE); Bernhard Goller, Penzberg (DE); Anja Limberg, Basel (CH); Petra Rueger, Penzberg (DE); Matthias Rueth, Penzberg (DE); Christine Schuell, Penzberg (DE); Mark Stahl, Tuebingen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/293,428

(22) PCT Filed: Mar. 21, 2007
(86) PCT No.: PCT/EP07/02487

§ 371 Date: Sep. 18, 2008

(30) Foreign Application Priority Data
Mar. 23, 2006 (EP) 06006008.4

(51) Int. Cl. A61K 31/4188; C07D 487/04; A61K 31/4196; A61K 31/4439; A61K 31/506

(52) U.S. Cl. 514/256; 548/266.4; 548/253; 548/302.1; 546/273.1; 544/242; 514/383; 514/381; 514/394; 514/338

(57) ABSTRACT

Objects of the present invention are the compounds of formula I

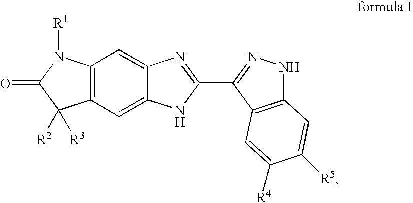

their pharmaceutically acceptable salts, enantiomeric forms, diastereoisomers and racemates, the preparation of the above-mentioned compounds, medicaments containing them and their manufacture, as well as the use of the above-mentioned compounds in the control or prevention of illnesses such as cancer.

The present invention relates to substituted indazole derivatives, to a process for their manufacture, pharmaceutical compositions containing them and their manufacture as well as the use of these compounds as pharmaceutically active agents.

### BACKGROUND OF THE INVENTION

Protein kinases regulate many different signaling processes by adding phosphate groups to proteins (Hunter, T., Cell 50 (1987) 823-829); particularly serine/threonine kinases phosphorylate proteins on the alcohol moiety of serine or threonine residues. The serine/threonine kinase family includes members that control cell growth, migration, differentiation, gene expression, muscle contraction, glucose metabolism, cellular protein synthesis, and regulation of the cell cycle.

The Aurora kinases are a family of serine/threonine kinases that are believed to play a key role in the protein phosphorylation events that are essential for the completion of essential mitotic events. The Aurora kinase family is made up of three key members: Aurora A, B and C (also known as Aurora-2, Aurora-1 and Aurora-3 respectively). Aurora-1 and Aurora-2 are described in U.S. Pat. No. 6,207,401 of Sugen and in related patents and patent applications, e.g. EP 0 868 519 and EP 1 051 500.

For Aurora A there is increasing evidence that it is a novel proto-oncogene. Aurora A gene is amplified and transcript/protein is highly expressed in a majority of human tumor cell lines and primary colorectal, breast and other tumors. It has been shown that Aurora A overexpression leads to genetic instability shown by amplified centrosomes and significant increase in aneuploidy and transforms Rat1 fibroblasts and mouse NIH3T3 cells in vitro. Aurora A-transformed NIH3T3 cells grow as tumors in nude mice (Bischoff, J. R., and Plowman, G. D., Trends Cell Biol. 9 (1999) 454-459; Giet, R., and Prigent, C., J. Cell Sci. 112 (1999) 3591-3601; Nigg, E. A., Nat. Rev. Mol. Cell. Biol. 2 (2001) 21-32; Adams, R. R., et al., Trends Cell Biol. 11 (2001) 49-54). Moreover, amplification of Aurora A is associated with aneuploidy and aggressive clinical behavior (Sen, S., et al., J. Natl. Cancer Inst. 94 (2002) 1320-1329) and amplification of its locus correlates with poor prognosis for patients with node-negative breast cancer (Isola, J. J., et al., Am. J. Pathology 147 (1995) 905-911). For these reasons it is proposed that Aurora A overexpression contributes to cancer phenotype by being involved in chromosome segregation and mitotic checkpoint control.

Human tumor cell lines depleted of Aurora A transcripts arrest in mitosis. Accordingly, the specific inhibition of Aurora kinase by selective inhibitors is recognized to stop uncontrolled proliferation, re-establish mitotic checkpoint control and lead to apoptosis of tumor cells. In a xenograft model, an Aurora inhibitor therefore slows tumor growth and induces regression (Harrington, E. A., et al., Nat. Med. 10 (2004) 262-267).

Low molecular weight inhibitors for protein kinases are widely known in the state of the art. For Aurora inhibition such inhibitors are based on i.e. quinazoline derivatives as claimed in the following patents and patent applications: WO 00/44728; WO 00/47212; WO 01/21594; WO 01/21595; WO 01/21596; WO 01/21597; WO 01/77085; WO 01/55116; WO 95/19169; WO 95/23141; WO 97/42187; WO 99/06396; pyrazole derivatives as claimed in the following patents and patent applications: WO 02/22601; WO 02/22603; WO 02/22604; WO 02/22605; WO 02/22606; WO 02/22607; WO 02/22608; WO 02/50065; WO 02/50066; WO 02/057259; WO 02/059112; WO 02/059111; WO 02/062789; WO 02/066461; WO 02/068415.

Some tricyclic heterocycles or related compounds are known as inhibitors of erythrocyte aggregation from Mertens, A., et al., J. Med. Chem. 30 (1987) 1279-1287; von der Saal, W., et al., J. Med. Chem. 32 (1989) 1481-1491; U.S. Pat. No. 4,666,923A; U.S. Pat. No. 4,695,567A; U.S. Pat. No. 4,863,945A and U.S. Pat. No. 4,954,498A.

WO 03/035065 relates to benzimidazole derivatives as kinase inhibitors, especially as inhibitors against KDR, SYK and ITK tyrosine kinases. WO 01/02369 and WO 01/53268 relate to indazole derivatives as kinase inhibitors, especially as inhibitors against VGEF, LCK, FAK, TEK, CHK-1 and CDKs, with antiproliferative activity.

### SUMMARY OF THE INVENTION

The present invention relates to tricyclic aminopyrazole derivatives of the general formula I,

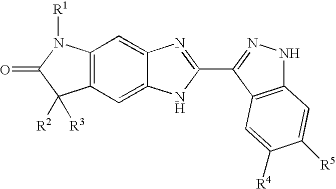

wherein
R¹ is alkyl;
R² and R³ are alkyl;
one of R⁴ and R⁵ is a) -X-heteroaryl, wherein the heteroaryl is optionally substituted one to three times by alkyl, alkyl-C(O)—, alkoxy, fluorinated alkyl, fluorinated alkoxy, cyano, nitro, amino, alkylamino, dialkylamino or halogen;
   b) -Y-phenyl,
   wherein the phenyl is optionally substituted one to three times by alkyl, alkyl-C(O)—, carboxy, alkyl-NHC(O)—, alkoxy, fluorinated alkyl, fluorinated alkoxy, cyano, hydroxy, nitro, amino, alkylamino, dialkylamino, alkyl-C(O)NH—, alkyl-S(O)₂NH—, halogen, 2,4-dioxa-pentan-1,5-diyl or 2,5-dioxa-hexan-1,6-diyl;
   or wherein the phenyl is substituted once by phenyl; or
   c) -Z-cycloalkyl;
and the other of R⁴ and R⁵ is hydrogen;
X is a single bond, —CH═CH— or —C≡C—;
Y is a single bond, —CH═CH— or —C≡C—;
Z is —CH═CH—;
and all pharmaceutically acceptable salts thereof.

The compounds according to this invention show activity as Aurora family kinase inhibitors, especially as Aurora A kinase inhibitors, and may therefore be useful for the treatment of diseases mediated by said kinase. Aurora A inhibition leads to cell cycle arrest in the G2 phase of the cell cycle and exerts an antiproliferative effect in tumor cell lines. This indicates that Aurora A inhibitors may be useful in the treatment of i.e. hyperproliferative diseases such as cancer and in particular colorectal, breast, lung, prostate, pancreatic, gastric, bladder, ovarian, melanoma, neuroblastoma, cervical, kidney or renal cancers, leukemias or lymphomas. Treatment of acute-myelogenous leukemia (AML, acute lymphocytic leukemia (ALL) and gastrointestinal stromal tumor (GIST) is included.

Objects of the present invention are the compounds of formula I and their tautomers, pharmaceutically acceptable salts, enantiomeric forms, diastereoisomers and racemates, their use as Aurora kinase inhibitors, the preparation of the above-mentioned compounds, medicaments containing them and their manufacture as well as the use of the above-mentioned compounds in treatment, control or prevention of illnesses, especially of illnesses and disorders as mentioned above like tumors or cancer (e.g. colorectal, breast, lung, prostate, pancreatic, gastric, bladder, ovarian, melanoma, neuroblastoma, cervical, kidney or renal cancers, leukemias or lymphomas) or in the manufacture of corresponding medicaments.

### DETAILED DESCRIPTION OF THE INVENTION

The term “alkyl” as used herein means a saturated, straight-chain or branched-chain hydrocarbon containing from 1 to 6 carbon atoms, preferably from 1 to 4 carbon atoms, such as methyl, ethyl, n-propyl, isopropyl, n-butyl, 2-butyl, t-butyl, n-pentyl, n-hexyl.

The term “alkoxy” as used herein means an alkyl-O-group wherein the alkyl is defined as above.

The term “alkylamino” as used herein means an alkyl-NH— group wherein the alkyl is defined as above.

The term “dialkylamino” as used herein means an (alkyl)₂N— group wherein the alkyl is defined as above.

The term “halogen” as used herein means fluorine, chlorine or bromine, preferably fluorine or chlorine.

The term “fluorinated alkyl” as used herein means an alkyl group as defined above which is substituted one or several times, preferably one to six and more preferably one to three times, by fluorine. Examples are difluoromethyl, trifluoromethyl, 2,2,2-trifluoroethyl, perfluorethyl, and the like, preferably trifluoromethyl.

The term “fluorinated alkoxy” as used herein means an alkoxy group as defined above which is substituted one or several times, preferably one to six and more preferably one to three times, by fluorine. Examples are difluoromethoxy, trifluoromethoxy, 2,2,2-trifluoroethoxy, perfluoroethoxy and the like, preferably trifluoromethoxy.

The term “cycloalkyl” as used herein means a monocyclic saturated hydrocarbon ring with 3 to 7, preferably 3 to 6, ring atoms. Examples of such saturated carbocyclic groups are e.g. cyclopropyl, cyclobutyl, cyclopentyl, cyclohexyl or cycloheptyl, preferably cyclopentyl or cyclohexyl.

The term “heteroaryl” means a mono- or bicyclic aromatic ring with 5 to 10, preferably 5 to 6, ring atoms, which contains up to 3, preferably 1 or 2 heteroatoms selected independently from N, O or S and the remaining ring atoms being carbon atoms. Examples of such heteroaryl groups include pyrrolyl, imidazolyl, pyrazolyl, triazolyl, tetrazolyl, furanyl, oxazolyl, isoxazolyl, thienyl, thiazolyl, pyridyl, pyrimidyl, pyridazinyl, pyrazinyl, indolyl, indazolyl, benzimidazolyl, benzothiophenyl, benzofuranyl, quinolyl, isoquinolyl, quinazolinyl, quinoxalinyl and the like, preferably pyrazolyl, triazolyl, tetrazolyl, thienyl, pyridyl or pyrimidyl.

If the heteroaryl group of -X-heteroaryl in the definition of R⁴ and R⁵ is substituted, such heteroaryl group is substituted preferably one or two times.

If the phenyl group of -Y-phenyl in the definition of R⁴ and R⁵ is substituted, such phenyl group is substituted preferably one or two times.

If the phenyl group of -Y-phenyl in the definition of R⁴ and R⁵ is substituted by 2,4-dioxa-pentan-1,5-diyl or 2,5-dioxa-hexan-1,6-diyl, it is substituted preferably once by 2,4-dioxa-pentan-1,5-diyl or 2,5-dioxa-hexan-1,6-diyl and forms together with the 2,4-dioxa-pentan-1,5-diyl or the 2,5-dioxa-hexan-1,6-diyl substituent a benzo[1,3]dioxolyl or a 2,3-dihydro-benzo[1,4]dioxinyl moiety.

As used herein, in relation to mass spectrometry (MS) the term “ESI+” refers to positive electrospray ionization mode, the term “ESI−” refers to negative electrospray ionization mode, the term “API+” refers to positive atmospheric pressure ionization mode and the term “API−” refers to negative atmospheric pressure ionization mode.

As used herein, in relation to nuclear magnetic resonance (NMR) the term “DMSO” refers to deuterated dimethylsulfoxide.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” or a “pharmaceutically acceptable adjuvant” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

The compounds of formula I can exist in different tautomeric forms and in variable mixtures thereof. All tautomeric forms of the compounds of formula I and mixtures thereof are an objective of the invention. For example, the imidazole part of the tricyclic ring system of formula I can exist in two tautomeric forms as shown here below:

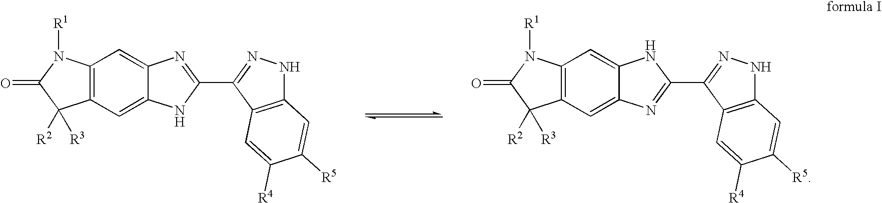

One embodiment of invention are the compounds according to formula I, wherein
one of R⁴ and R⁵ is a) -X-heteroaryl, wherein the heteroaryl is optionally substituted one to three times, preferably once or twice, by alkyl or alkoxy;
   b) -Y-phenyl,
   wherein the phenyl is optionally substituted one to three times, preferably once or twice, by alkyl, alkyl-C(O)—, alkoxy, fluorinated alkyl, nitro, dialkylamino, halogen or 2,4-dioxa-pentan-1,5-diyl; or wherein the phenyl is substituted once by phenyl; or
   c) -Z-cycloalkyl;
and the other of R⁴ and R⁵ is hydrogen;
X is a single bond;
Y is a single bond, —CH═CH— or —C≡C—; and
Z is —CH═CH—.

Another embodiment of invention are the compounds according to formula I, wherein
one of R⁴ and R⁵ is -X-heteroaryl, wherein the heteroaryl is optionally substituted one to three times by alkyl or alkoxy;
and the other of R⁴ and R⁵ is hydrogen;

Another embodiment of invention are the compounds according to formula I, wherein
one of R⁴ and R⁵ is -X-heteroaryl, wherein the heteroaryl is optionally substituted one to three times by alkyl or alkoxy;
and the other of R⁴ and R⁵ is hydrogen; and
X is a single bond.

Such compounds, for example, may be selected from the group consisting of:
5-Ethyl-7,7-dimethyl-2-[5-(1H-[1,2,4]triazol-3-yl)-1H-indazol-3-yl]-5,7-dihydro-3H-imidazo[4,5-f]indol-6-one;
5-Ethyl-7,7-dimethyl-2-[6-(1H-[1,2,4]triazol-3-yl)-1H-indazol-3-yl]-5,7-dihydro-3H-imidazo[4,5-f]indol-6-one;
5-Ethyl-7,7-dimethyl-2-[5-(1H-tetrazol-5-yl)-1H-indazol-3-yl]-5,7-dihydro-3H-imidazo[4,5-f]indol-6-one;
5-Ethyl-7,7-dimethyl-2-(6-thiophen-3-yl-1H-indazol-3-yl)-5,7-dihydro-3H-imidazo[4,5-f]indol-6-one;
5-Ethyl-7,7-dimethyl-2-[6-(1-methyl-1H-pyrazol-4-yl)-1H-indazol-3-yl]-5,7-dihydro-3H-imidazo[4,5-f]indol-6-one;
5-Ethyl-7,7-dimethyl-2-(6-pyridin-3-yl-1H-indazol-3-yl)-5,7-dihydro-3H-imidazo[4,5-f]indol-6-one;
5-Ethyl-2-[6-(6-methoxy-pyridin-3-yl)-1H-indazol-3-yl]-7,7-dimethyl-5,7-dihydro-3H-imidazo[4,5-f]indol-6-one;
5-Ethyl-7,7-dimethyl-2-(6-pyridin-4-yl-1H-indazol-3-yl)-5,7-dihydro-3H-imidazo[4,5-f]indol-6-one;
5-Ethyl-7,7-dimethyl-2-(6-thiophen-2-yl-1H-indazol-3-yl)-5,7-dihydro-3H-imidazo[4,5-f]indol-6-one;
5-Ethyl-2-[5-(6-methoxy-pyridin-3-yl)-1H-indazol-3-yl]-7,7-dimethyl-5,7-dihydro-3H-imidazo[4,5-f]indol-6-one; compound with acetic acid;
5-Ethyl-7,7-dimethyl-2-(5-thiophen-3-yl-1H-indazol-3-yl)-5,7-dihydro-3H-imidazo[4,5-f]indol-6-one; compound with acetic acid;
5-Ethyl-7,7-dimethyl-2-[5-(1-methyl-1H-pyrazol-4-yl)-1H-indazol-3-yl]-5,7-dihydro-3H-imidazo[4,5-f]indol-6-one; compound with acetic acid;
5-Ethyl-7,7-dimethyl-2-(5-pyridin-3-yl-1H-indazol-3-yl)-5,7-dihydro-3H-imidazo[4,5-f]indol-6-one;
5-Ethyl-7,7-dimethyl-2-(6-pyrimidin-5-yl-1H-indazol-3-yl)-5,7-dihydro-3H-imidazo[4,5-f]indol-6-one;
5-Ethyl-7,7-dimethyl-2-(6-pyridin-2-yl-1H-indazol-3-yl)-5,7-dihydro-3H-imidazo[4,5-f]indol-6-one;
5-Ethyl-7,7-dimethyl-2-(5-pyrimidin-5-yl-1H-indazol-3-yl)-5,7-dihydro-3H-imidazo[4,5-f]indol-6-one;
5-Ethyl-7,7-dimethyl-2-(5-pyridin-2-yl-1H-indazol-3-yl)-5,7-dihydro-3H-imidazo[4,5-f]indol-6-one; and
5-Ethyl-7,7-dimethyl-2-[6-(1H-pyrazol-4-yl)-1H-indazol-3-yl]-5,7-dihydro-3H-imidazo[4,5-f]indol-6-one.

Another embodiment of invention are the compounds according to formula I, wherein
one of R⁴ and R⁵ is -Y-phenyl,
   wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl;
and the other of R⁴ and R⁵ is hydrogen.

Another embodiment of invention are the compounds according to formula I, wherein
one of R⁴ and R⁵ is -Y-phenyl,
   wherein the phenyl is optionally substituted one to three times by alkyl-C(O)—, carboxy, alkoxy, nitro, dialkylamino or halogen; or wherein the phenyl is substituted once by phenyl;
and the other of R⁴ and R⁵ is hydrogen; and
Y is a single bond.

Such compounds, for example, may be selected from the group consisting of:
2-[6-(4-Dimethylamino-phenyl)-1H-indazol-3-yl]-5-ethyl-7,7-dimethyl-5,7-dihydro-3H-imidazo[4,5-f]indol-6-one;
2-[6-(4-Acetyl-phenyl)-1H-indazol-3-yl]-5-ethyl-7,7-dimethyl-5,7-dihydro-3H-imidazo[4,5-f]indol-6-one;
4-[3-(5-Ethyl-7,7-dimethyl-6-oxo-3,5,6,7-tetrahydro-imidazo[4,5-f]indol-2-yl)-1H-indazol-6-yl]-benzoic acid;
2-(6-Benzo[1,3]dioxol-5-yl-1H-indazol-3-yl)-5-ethyl-7,7-dimethyl-5,7-dihydro-3H-imidazo[4,5-f]indol-6-one;
2-[6-(3-Dimethylamino-phenyl)-1H-indazol-3-yl]-5-ethyl-7,7-dimethyl-5,7-dihydro-3H-imidazo[4,5-f]indol-6-one;
5-Ethyl-7,7-dimethyl-2-[6-(3-nitro-phenyl)-1H-indazol-3-yl]-5,7-dihydro-3H-imidazo[4,5-f]indol-6-one;
2-[5-(4-Dimethylamino-phenyl)-1H-indazol-3-yl]-5-ethyl-7,7-dimethyl-5,7-dihydro-3H-imidazo[4,5-f]indol-6-one;
2-[5-(3-Dimethylamino-phenyl)-1H-indazol-3-yl]-5-ethyl-7,7-dimethyl-5,7-dihydro-3H-imidazo[4,5-f]indol-6-one;
2-(5-Benzo[1,3]dioxol-5-yl-1H-indazol-3-yl)-5-ethyl-7,7-dimethyl-5,7-dihydro-3H-imidazo[4,5-f]indol-6-one; compound with acetic acid;
5-Ethyl-7,7-dimethyl-2-(6-phenyl-1H-indazol-3-yl)-5,7-dihydro-3H-imidazo[4,5-f]indol-6-one; and
2-[6-(3,5-Dimethoxy-phenyl)-1H-indazol-3-yl]-5-ethyl-7,7-dimethyl-5,7-dihydro-3H-imidazo[4,5-f]indol-6-one.

Another embodiment of invention are the compounds according to formula I, wherein
one of R⁴ and R⁵ is -Y-phenyl,
   wherein the phenyl is optionally substituted one to three times by alkoxy, fluorinated alkyl, nitro or halogen; or wherein the phenyl is substituted once by phenyl;
and the other of R⁴ and R⁵ is hydrogen; and
Y is —CH═CH—.

Such compounds, for example, may be selected from the group consisting of:
5-Ethyl-7,7-dimethyl-2-[6-((E)-styryl)-1H-indazol-3-yl]-5,7-dihydro-3H-imidazo[4,5-f]indol-6-one;
5-Ethyl-2-{6-[(E)-2-(4-fluoro-phenyl)-vinyl]-1H-indazol-3-yl}-7,7-dimethyl-5,7-dihydro-3H-imidazo[4,5-f]indol-6-one;
2-[6-((E)-2-Biphenyl-4-yl-vinyl)-1H-indazol-3-yl]-5-ethyl-7,7-dimethyl-5,7-dihydro-3H-imidazo[4,5-f]indol-6-one;
5-Ethyl-2-{6-[(E)-2-(4-methoxy-phenyl)-vinyl]-1H-indazol-3-yl}-7,7-dimethyl-5,7-dihydro-3H-imidazo[4,5-f]indol-6-one;
5-Ethyl-7,7-dimethyl-2-{6-[(E)-2-(4-trifluoromethyl-phenyl)-vinyl]-1H-indazol-3-yl}-5,7-dihydro-3H-imidazo[4,5-f]indol-6-one;
2-{6-[(E)-2-(4-Chloro-phenyl)-vinyl]-1H-indazol-3-yl}-5-ethyl-7,7-dimethyl-5,7-dihydro-3H-imidazo[4,5-f]indol-6-one;
5-Ethyl-2-{6-[(E)-2-(3-fluoro-phenyl)-vinyl]-1H-indazol-3-yl}-7,7-dimethyl-5,7-dihydro-3H-imidazo[4,5-f]indol-6-one; and
5-Ethyl-7,7-dimethyl-2-{6-[(E)-2-(3-nitro-phenyl)-vinyl]-1H-indazol-3-yl}-5,7-dihydro-3H-imidazo[4,5-f]indol-6-one; compound with acetic acid.

Another embodiment of invention are the compounds according to formula I, wherein
one of R⁴ and R⁵ is -Y-phenyl,
   wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl;
and the other of R⁴ and R⁵ is hydrogen; and
Y is —C≡C—.

Such a compound is for example:
5-Ethyl-7,7-dimethyl-2-(6-phenylethynyl-1H-indazol-3-yl)-5,7-dihydro-3H-imidazo[4,5-f]indol-6-one.

Another embodiment of invention are the compounds according to formula I, wherein
one of R⁴ and R⁵ is -Z-cycloalkyl;
and the other of R⁴ and R⁵ is hydrogen.

Such a compound is for example:
2-[6-((E)-2-Cyclohexyl-vinyl)-1H-indazol-3-yl]-5-ethyl-7,7-dimethyl-5,7-dihydro-3H-imidazo[4,5-f]indol-6-one.

Another embodiment of invention are the compounds according to formula I, wherein
R⁴ is a) -X-heteroaryl, wherein the heteroaryl is optionally substituted one to three times by alkyl or alkoxy;
   b) -Y-phenyl,
   wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl; or
   c) -Z-cycloalkyl;
R⁵ is hydrogen;
X is a single bond;
Y is a single bond, —CH═CH— or —C≡C—; and
Z is —CH═CH—.

Another embodiment of invention are the compounds according to formula I, wherein
R⁴ is -X-heteroaryl, wherein the heteroaryl is optionally substituted one to three times by alkyl or alkoxy;
R⁵ is hydrogen; and
X is a single bond.

Another embodiment of invention are the compounds according to formula I, wherein
R⁴ is -Y-phenyl,
   wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl;
R⁵ is hydrogen; and
Y is a single bond, —CH═CH— or —C≡C—.

Another embodiment of invention are the compounds according to formula I, wherein
R⁴ is -Z-cycloalkyl;
R⁵ is hydrogen; and
Z is —CH═CH—.

Another embodiment of invention are the compounds according to formula I, wherein
R⁵ is a) -X-heteroaryl, wherein the heteroaryl is optionally substituted one to three times by alkyl or alkoxy;
   b) -Y-phenyl,
   wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl; or
   c) -Z-cycloalkyl;
R⁴ is hydrogen;
X is a single bond;
Y is a single bond, —CH═CH— or —C≡C—; and
Z is —CH═CH—.

Another embodiment of invention are the compounds according to formula I, wherein
R⁵ is -X-heteroaryl, wherein the heteroaryl is optionally substituted one to three times by alkyl or alkoxy;
R⁴ is hydrogen; and
X is a single bond.

Another embodiment of invention are the compounds according to formula I, wherein
R⁵ is -Y-phenyl,
   wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl;
R⁴ is hydrogen; and
Y is a single bond, —CH═CH— or —C≡C—.

Another embodiment of invention are the compounds according to formula I, wherein
R⁵ is -Z-cycloalkyl;
R⁴ is hydrogen; and
Z is —CH═CH—.

Another embodiment of invention is a process for the preparation of the compounds of formula I by
a) reacting a compound of formula V,

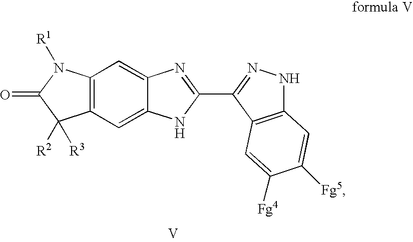

wherein R¹, R² and R³ have the significance given above for formula I, one of Fg⁴ and Fg⁵ represents a functional group selected from bromine, iodine, boronic acids or boronic acid esters and the other of Fg⁴ and Fg⁵ is hydrogen,
with a compound of formula VIa or VIb,

R⁴-G formula VIa

or

R⁵-G formula VIb,
wherein R⁴ and R⁵ have the significance given above for formula I and G represents a functional group selected from the group consisting of: hydrogen, bromine, iodine, boronic acids and boronic acid esters,
with the proviso that if G is bromine or iodine, Fg⁴ or Fg⁵ is boronic acid or a boronic acid ester, and if G is hydrogen, boronic acid or a boronic acid ester, Fg⁴ or Fg⁵ is bromine or iodine,
to give the compounds of formula I

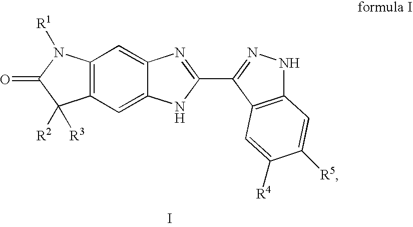

wherein R¹, R², R³, R⁴ and R⁵ have the significance given above for formula I,
b) isolating the compounds of formula I; and
c) if desired, converting the compounds of formula I into their pharmaceutically acceptable salts.

The compounds of formula I, or a pharmaceutically acceptable salt thereof, which are subject of the present invention, may be prepared by any process known to be applicable to the preparation of chemically-related compounds. Such processes, when used to prepare a compound of the formula I, or a pharmaceutically-acceptable salt thereof, are illustrated by the following representative schemes 1 to 7 and examples in which, unless otherwise stated, R¹, R², R³, R⁴ and R⁵ have the significance given herein before for formula I. Necessary starting materials are either commercially available or they may be obtained by standard procedures of organic chemistry. The preparation of such starting materials is described within the accompanying examples or in the literature cited below with respect to scheme 1 to 7. Alternatively necessary starting materials are obtainable by analogous procedures to those illustrated which are within the ordinary skill of an organic chemist.

One route for the preparation of compounds of formula I starts from the diamines of formula II

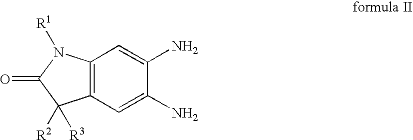

In formula II, R¹, R² and R³ have the significance as given above for formula I.

The synthesis of diamines of formula II or precursors thereof is described in Mertens, A., et al., J. Med. Chem. 30 (1987) 1279-1287; von der Saal, W., et al., J. Med. Chem. 32 (1989) 1481-1491; U.S. Pat. No. 4,666,923A, U.S. Pat. No. 4,695,567A, U.S. Pat. No. 4,863,945A, U.S. Pat. No. 4,985,448A and DE 34 10 168. For instance, the diamines of formula II, can be synthesized as shown in Scheme 1a:

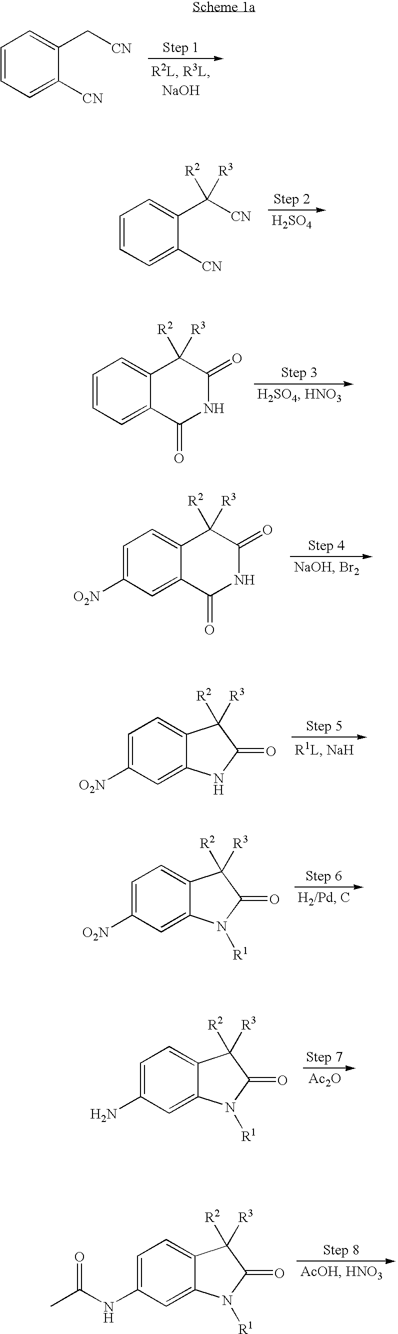

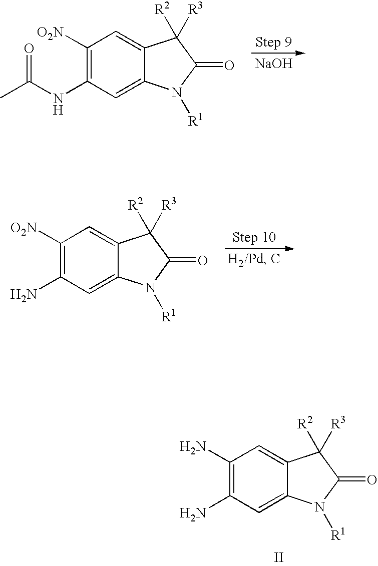

In scheme 1a, R¹, R² and R³ have the significance as given above for formula I, except that R¹ is not hydrogen, and L represents a leaving group as e.g. iodine, bromine, chlorine, triflate and the like.

In an alternative procedure diamines of formula II can be obtained by an alkylation of diamines of formula III as shown in scheme 1b. Diamines of formula III can be synthesized according to scheme 1 under omission of step 5.

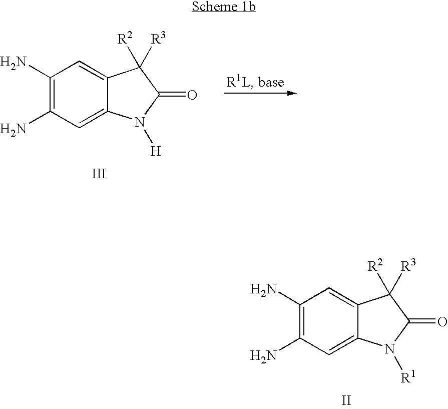

In scheme 1b, R¹, R² and R³ have the significance as given above for formula I, except that R¹ is not hydrogen, and L represents a leaving group as e.g. iodine, bromine, chlorine, triflate and the like. The alkylation reaction is typically carried out in the presence of a base such as sodium hydride, potassium hydride and the like, especially sodium hydride, in inert solvents such as dimethylformamide (DMF), N-methyl-pyrrolidinone (NMP), tetrahydrofuran and the like.

Diamines of formula II are subsequently employed in the formation of the imidazole ring system of formula I. Different synthetic pathways for this cyclization are described in the literature (e.g. see Mertens, A., et al., J. Med. Chem. 30 (1987) 1279-1287 and U.S. Pat. No. 4,695,567A).

For example, as shown in Scheme 2, diamines of formula II can be reacted with carboxylic acids (indazole compounds of formula IV wherein A is hydroxy), acid chlorides (indazole compounds of formula IV wherein A is chlorine), aldehydes (indazole compounds of formula IV wherein A is hydrogen), methyl carboxylates (indazole compounds of formula IV wherein A is methoxy) or activated esters (indazole compounds of formula IV wherein A is e.g. hydroxybenzotriazole). For detailed procedures see Mertens, A., et al., J. Med. Chem. 30 (1987) 1279-1287 and U.S. Pat. No. 4,695,567A.

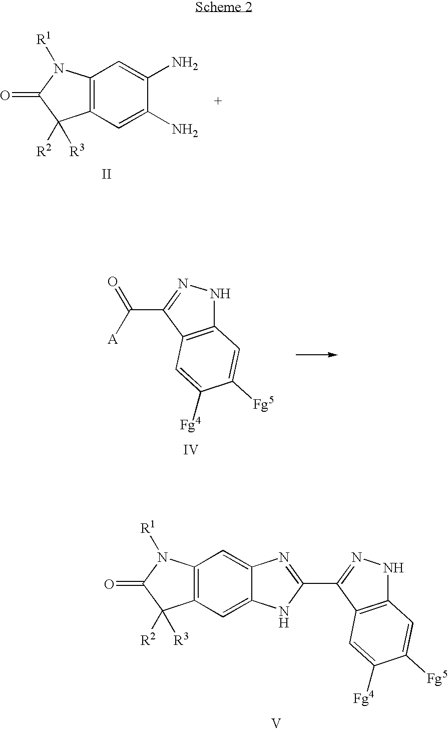

In scheme 2, R¹, R² and R³ have the significance as given above for formula I and A is hydroxy, chlorine, hydrogen, methoxy or e.g. hydroxybenzotriazole. One of the substituents Fg⁴ and Fg⁵ is a functional group suitable for conversion into R⁴ and R⁵ and the other of Fg⁴ and Fg⁵ is hydrogen. If Fg⁴ or Fg⁵ is a functional group suitable for conversion into R⁴ or R⁵ such functional group is selected from the group consisting of: carboxy, cyano, bromine, iodine, triflate, —ZnCl, boronic acids, boronic acid esters (e.g. boronic acid pinacolesters) and trialkylstannanes (e.g. Me₃Sn, Bu₃Sn). Preferably such functional group is selected from the group consisting of: carboxy, cyano, bromine, iodine, boronic acids and boronic acid esters (e.g. boronic acid pinacolesters). Examples for the conversion into R⁴ and R⁵ (which have the meaning as defined above for formula I) are described in schemes 5-7.

Indazoles of formula IV are either commercially available or they can be prepared by different synthetic routes according to the nature of “A”. If “A” is hydroxy the corresponding 3-indazolecarboxylic acids are named IVa and can be manufactured e.g. as shown in the following scheme 3.

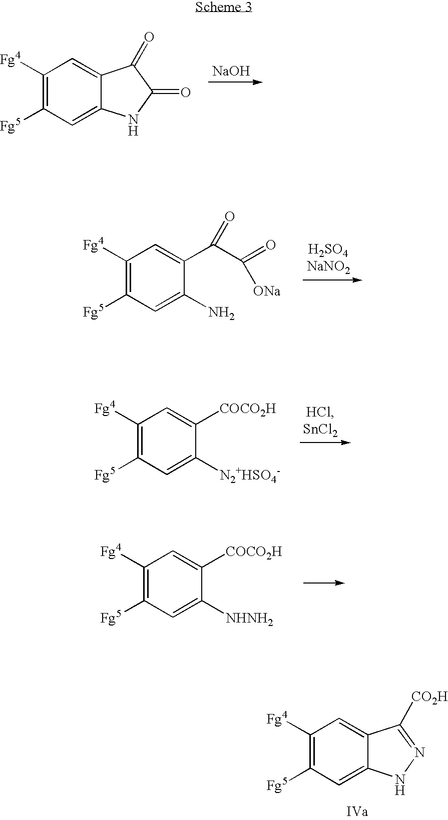

In scheme 3, Fg⁴ and Fg⁵ have the significance as given above for scheme II. As described in Snyder, H. R., et al., J. Am. Chem. Soc. 74 (1952) 2009-2012, 3-indazolecarboxylic acids of formula IIIa can be prepared from isatins by basic ring opening, followed by diazotation of the amino group, reduction to the hydrazine and condensation to give the desired indazole.

The necessary isatins are either commercially available or may be obtained by standard procedures of organic chemistry, e.g. by reaction of the corresponding aniline with oxalylchloride. The reaction starts with an N-acylation, followed by an intramolecular acylation which can be catalyzed by Lewis acids. (e.g. Piggott, M. J. and Wege, D., Australian Journal of Chemistry 53 (2000) 749-754; March, J., Advanced Organic Chemistry 4th ed., John Wiley & Sons, New York (1992) 539-542) More often the corresponding aniline is reacted with chloral hydrate (2,2,2-trichlor-1,1-ethanediol) and hydroxylamine (hydrochloride) (via the hydroxyiminoacetamides) in a cyclization reaction to the desired isatins (e.g. Sheibley, F. E., and McNulty, J. S., J. Org. Chem. 21 (1956) 171-173; Lisowski, V., et al., J. Org. Chem. 65 (2000) 4193-4194).

If “A” is hydrogen, the corresponding 1H-indazole-3-carbaldehydes are named IVb and can be manufactured e.g. as shown in the following scheme 4.

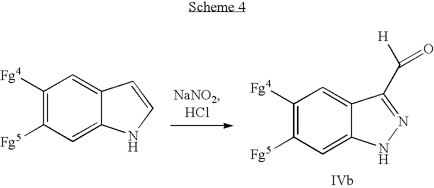

In scheme 4, Fg⁴ and Fg⁵ have the significance as given above for scheme II. The compounds of formula IVb can be synthesized from suitably substituted indoles by treatment with NaNO₂/HCl as described e.g. in Sall, D. J., et al., J. Med. Chem. 40 (1997) 2843-2857.

Compounds of the formula I wherein R⁴ or R⁵ have the meaning as defined above can be prepared e.g. by a palladium catalyzed coupling reaction as shown in scheme 5 between a compounds of formula V wherein R¹, R² and R³ have the meaning as defined above and Fg⁴ and Fg⁵ represent a functional group suitable for coupling reactions like bromine, iodine, triflate, —ZnCl, boronic acids, boronic acid pinacolesters and trialkylstannanes (e.g. Me₃Sn, Bu₃Sn) and a compound of formula VIa or VIb:

R⁴-G formula VIa

or

R⁵-G formula VIb

wherein R⁴ and R⁵ have the meaning as defined above and G represents a functional group suitable for coupling reactions, and compatible with Fg, as described above. G is selected from the group consisting of: hydrogen, bromine, iodine, triflate, —ZnCl, boronic acids, boronic acid esters (e.g. boronic acid pinacolesters) and trialkylstannanes (e.g. Me₃Sn, Bu₃Sn). Preferably G is selected from the group consisting of: hydrogen, bromine, iodine, boronic acids and boronic acid esters.

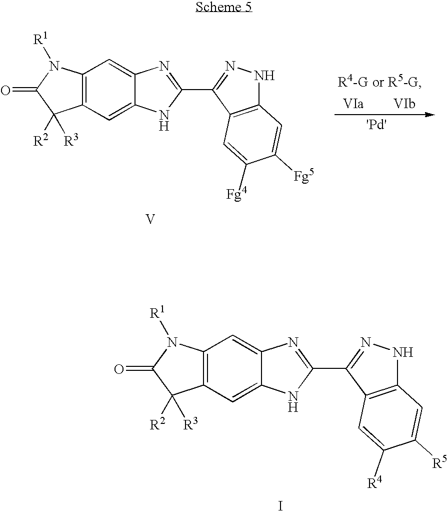

This reaction may be for example, but not limited to, a Suzuki type palladium catalyzed cross coupling reaction (G is boronic acid, boronic acid pinacolester etc. and Fg is bromine or iodine or Fg is boronic acids, boronic acid pinacolester etc. and G is bromine or iodine; see e.g. Miyaura, N., et al., Chem. Rev. 95 (1995) 2457; Miyaura, N., et al., Synth. Commun., 11 (1981) 513), a Negishi type reaction (G is ZnCl etc. and Fg is bromine or iodine or Fg is ZnCl etc. and G is bromine or iodine; see e.g. Negishi, E., et al., J. Org. Chem. 42 (1977) 1821) or a Stille type reaction (G is trialkylstannane e.g. Me₃Sn, Bu₃Sn and Fg is triflate, bromine or iodine or Fg is trialkylstannane e.g. Me₃Sn, Bu₃Sn and G is triflate, bromine or iodine; see e.g. Stille, J. K., Angew. Chem. 1986, 98, 504).

The intermediates of formulas V wherein Fg is a boronic acid, a boronic acid pinacolesters or trialkylstannane etc., can be obtained for example from the corresponding halogenides (Fg is bromine or iodine) by standard procedures of organic chemistry. For example compounds of formula V wherein Fg is a boronic acid pinacolester can be prepared from the bromide by a palladium catalyzed (e.g. PdCl₂(dppf)-CH₂Cl₂-complex) coupling with pinacolboran or bis(pinacolato)diboron. For example compounds of formula V wherein Fg is trialkylstannane can be prepared from the bromide by a palladium catalyzed (e.g. PdCl₂(MeCN)₂-Komplex) coupling with hexa-alkylditin.

The palladium catalyzed coupling reaction may also be for example, but not limited to, of Sonogashira type (Fg is e.g. Br, I or OTf, G is hydrogen and R⁴ or R⁵ is a optionally substituted phenylethynyl or a optionally substituted heteroarylethynyl group; see e.g. Sonogashira, K., et al., Tetrahedron Lett. 16 (1975) 4467-4470; Sonogashira, K., J. Organomet. Chem. 653 (2002) 46-49).

The palladium catalyzed coupling reaction may also be for example, but not limited to, of Heck type (Fg is e.g. Br, I or OTf, G is hydrogen and R⁴ or R⁵ is a optionally substituted styryl group or a optionally substituted heteroarylethenyl group; see e.g. Heck, R. F., et al., J. Org. Chem. 37 (1972) 2320).

Compounds of formula I wherein R⁴ or R⁵ is a triazole are named Ia and can be prepared e.g. from the corresponding carboxylic acids (compounds of formula V wherein Fg⁴ or Fg⁵ is COOH, which are named Va) as shown in the following scheme 6 (see e.g. Ankersen, M., et al., Bioorg. Med. Chem. Lett. 7 (1997) 1293-1298 or Lin, Y., et al., J. Org. Chem. 44 (1979) 4160-4164):

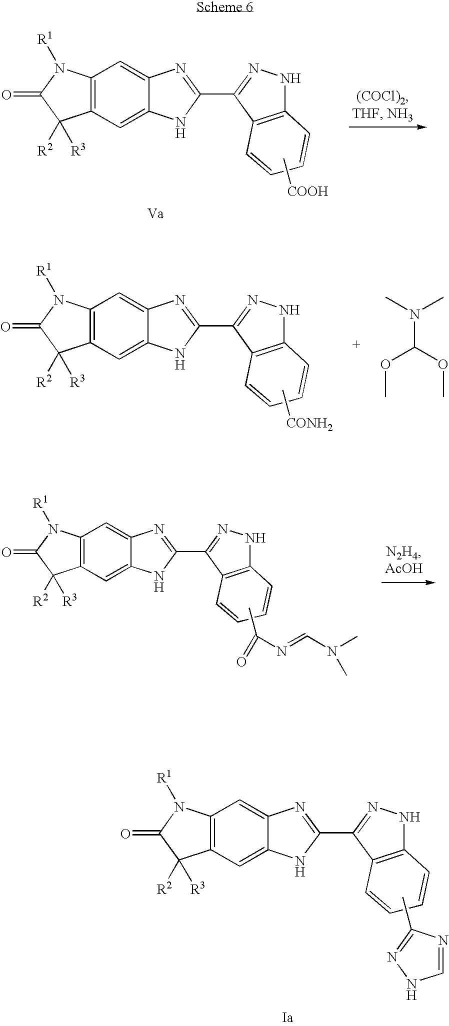

The carboxylic acids are converted to the amides which are reacted with N,N-dimethylformamide dimethyl acetal. The obtained acylamidines cyclize upon heating with hydrazine in glacial acetic acid to give the desired 1,2,4-triazoles.

Compounds of formula I wherein R⁴ or R⁵ is a tetrazole are named Ib and can be prepared e.g. from the corresponding nitriles (compounds of formula V wherein Fg⁴ or Fg⁵ is CN, which are named Vb) as shown in the following scheme 7 (see e.g. EP0512675A1 or Ankersen, M., et al., Bioorg. Med. Chem. Lett. 7 (1997) 1293-1298):

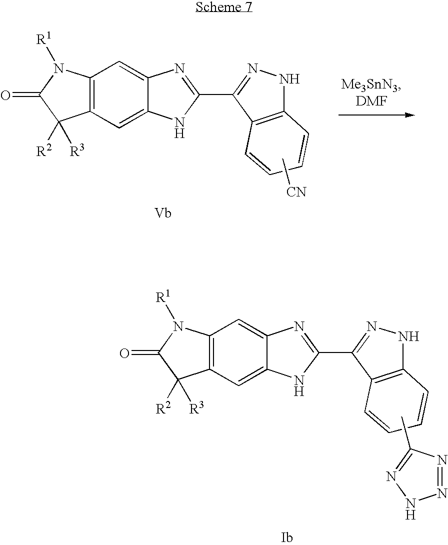

Cycloaddition of the nitriles with trimethyltin azide leads to formation of the tetrazole ring system.

Certain substituents on the groups R⁴ or R⁵ may not be inert to the conditions of the synthesis sequences described above and may require protection by standard protecting groups known in the art. For instance, an amino or hydroxyl group may be protected as an acetyl or tert-butyloxycarbonyl (BOC) derivative. Alternatively, some substituents may be derived from others at the end of the reaction sequence. For instance, a compound of formula I may be synthesized bearing a nitro-, a cyano, an ethoxycarbonyl, an ether, a sulfonic acid substituent on the group R⁴ or R⁵, which substituents are finally converted to an a) amino group—(e.g. by reduction of a nitro group, reduction of a cyano group or cleavage of a suitable amino protection group (for example by removal of a BOC group with trifluoroacetic acid (TFA))), b) alkylamino group—(e.g. by reductive amination of an amino group), c) dialkylamino group—(e.g. by alkylation of an amino group, reduction of an appropriate acylamino group with lithium aluminum hydride or Eschweiler-Clarke reaction with an appropriate amino or alkylamino group), d) acylamino group—(e.g. by amide formation from an amino group e.g. with appropriate acyl halides or with appropriate carboxylic acids after their activation with 1,1′-carbonyldiimidazole (CDI), 1-ethyl-3-[3-dimethylaminopropyl]-carbodiimide hydrochloride (EDC), etc.), e) alkylsulfonylamino group (e.g. by reaction of an amino group with sulfonyl chlorides), f) arylsulfonylamino group substituent (e.g. by reaction of an amino group with sulfonyl chlorides), g) hydroxyl group—(e.g. by cleavage of a suitable hydroxy protection group (e.g. hydrogenolytic removal of a benzyl ether or oxidative cleavage of a p-methoxy benzyl ether or fluoride assisted cleavage of silyl protecting group), h) ether group—(e.g. by Williamson's ether synthesis from a hydroxyl group), i) carboxamide group (e.g. by amide formation from a carboxylic acid group with appropriate amines after activation of the carboxylic acid group with CDI, EDC, etc. or conversion to an acyl chloride), or j) sulfonamide group by standard procedures.

Medicaments containing a compound of the present invention or a pharmaceutically acceptable salt thereof and a therapeutically inert carrier are an object of the present invention, as is a process for their production, which comprises bringing one or more compounds of the present invention and/or pharmaceutically acceptable salts and, if desired, one or more other therapeutically valuable substances into a galenical administration form together with one or more therapeutically inert carriers.

In accordance with the invention the compounds of the present invention as well as their pharmaceutically acceptable salts are useful in the control or prevention of illnesses. Based on their Aurora tyrosine kinase inhibition and/or their antiproliferative activity, said compounds are useful for the treatment of diseases such as cancer in humans or animals and for the production of corresponding medicaments. The dosage depends on various factors such as manner of administration, species, age and/or individual state of health.

An embodiment of the invention is a pharmaceutical composition, containing one or more compounds according to formula I, together with pharmaceutically acceptable excipients.

Another embodiment of the invention is a pharmaceutical composition containing one or more compounds of formula I as active ingredients together with pharmaceutically acceptable adjuvants for the treatment of diseases mediated by an inappropriate activation of Aurora family tyrosine kinases.

Another embodiment of the invention is a pharmaceutical composition, containing one or more compounds according to formula I as active ingredients together with pharmaceutically acceptable adjuvants for the inhibition of tumor growth.

Another embodiment of the invention is a pharmaceutical composition containing one or more compounds of formula I as active ingredients together with pharmaceutically acceptable adjuvants for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is a pharmaceutical composition containing one or more compounds of formula I as active ingredients together with pharmaceutically acceptable adjuvants for the treatment of acute-myelogenous leukemia (AML, acute lymphocytic leukemia (ALL) and gastrointestinal stromal tumor (GIST).

Another embodiment of the invention is the use of one or more compounds of formula I for the manufacture of medicaments for the treatment of diseases mediated by an inappropriate activation of Aurora family tyrosine kinases.

Another embodiment of the invention is the use of a compound according to formula I, for the manufacture of corresponding medicaments for the inhibition of tumor growth.

Another embodiment of the invention is the use of a compound according to formula I, for the manufacture of corresponding medicaments for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is the use of a compound according to formula I, for the manufacture of medicaments for the treatment of acute-myelogenous leukemia (AML, acute lymphocytic leukemia (ALL) and gastrointestinal stromal tumor (GIST).

Another embodiment of the invention is the use of the compounds of formula I as Aurora A tyrosine kinase inhibitors.

Another embodiment of the invention is the use of the compounds of formula I as anti-proliferating agents.

Another embodiment of the invention is the use of one or more compounds of formula I for the treatment of cancer.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g. Stahl, P. H., and Wermuth, G., (editors), Handbook of Pharmaceutical Salts, Verlag Helvetica Chimica Acta (VHCA), Züirich, (2002), or Bastin, R. J., et al., Organic Proc. Res. Dev. 4 (2000) 427-435.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases (HPLC: High Performance Liquid Chromatography) which are commercially available.

### Pharmacological Activity

The compounds of formula I and their pharmaceutically acceptable salts possess valuable pharmacological properties. It has been found that said compounds show activity as inhibitors of the Aurora kinase family and also show anti-proliferative activity. Consequently the compounds of the present invention are useful in the therapy and/or prevention of illnesses with known over-expression of kinases of the Aurora family, preferably Aurora A, especially in the therapy and/or prevention of illnesses mentioned above. The activity of the present compounds as inhibitors of the Aurora kinase family is demonstrated by the following biological assay:

### IC₅₀ Determination for Inhibitors of Aurora A

### Assay Principle

Aurora A is a serine threonine kinase involved in spindle assembly and chromosome segregation.

The assay is a typically ELISA-type assay where substrate (GST-Histone H3) is coupled to the assay-plate and is phosphorylated by the kinase. Phosphorylation is detected by a mouse anti-Phosphopeptid mAb and an HRP-labeled anti-mouse pAb. The assay is validated for IC₅₀-determination.

Kinase activities were measured by Enzyme-Linked Immunosorbent Assay (ELISA): Maxisorp 384-well plates (Nunc) were coated with recombinant fusion protein comprising residues 1-15 of HistoneH3 fused to the N-terminus of Glutathione-S-Transferase. Plates were then blocked with a solution of 1 mg/mL I-block (Tropix cat# T2015—highly purified form of casein) in phosphate-buffered saline. Kinase reactions were carried out in the wells of the ELISA plate by combining an appropriate amount of mutant Aurora A kinase with test compound and 30 μM ATP. The reaction buffer was 10× Kinase Buffer (Cell Signaling cat #9802) supplemented with 1 μg/mL I-block. Reactions were stopped after 40 minutes by addition of 25 mM EDTA. After washing, substrate phosphorylation was detected by addition of anti-phospho-Histone H3 (Ser 10) 6G3 mAb (Cell Signaling cat #9706) and sheep anti-mouse pAb-HRP (Amersham cat# NA931V), followed by colorimetric development with TMB (3,3′,5,5′-tetramethylbenzidine from Kirkegaard & Perry Laboratories). After readout of the absorbance, IC₅₀ values were calculated using a non-linear curve fit (XLfit software (ID Business Solution Ltd., Guilford, Surrey, UK)). The results are shown in Table 1.

TABLE 1
Results:

| | IC50 Aurora A kinase |
|---|---|
| Example No. | inhibition [μM] |
| 1 | 0.002 |
| 4 | 0.022 |
| 6 | 0.035 |
| 11 | 0.019 |
| 19 | 0.058 |
| 29 | 0.006 |
| 38 | 0.009 |
| 2, 3, 5, 7, 8, 10, 13, 14, 16, | 0.0001-0.100 |
| 17, 20, 23, 26, 27, 31, 32, | |
| 34, 37 | |

### Antiproliferative Activity

The activity of the present compounds as antiproliferative agents is demonstrated by the following biological assay:

### CellTiter-Glo™ Assay in HCT 116 Cells

The CellTiter-Glo™ Luminescent Cell Viability Assay (Promega) is a homogeneous method of determining the number of viable cells in culture based on quantitation of the ATP present, which signals the presence of metabolically active cells.

HCT 116 cells (human colon carcinoma, ATCC-No. CCl-247) were cultivated in RPMI 1640 medium with GlutaMAX™ I (Invitrogen, Cat-No. 61870-010), 2,5% Fetal Calf Serum (FCS, Sigma Cat-No. F4135 (FBS)); 100 Units/ml penicillin/100 μg/ml streptomycin (=Pen/Strep from Invitrogen Cat. No. 15140). For the assay the cells were seeded in 384 well plates, 1000 cells per well, in the same medium. The next day the test compounds were added in various concentrations ranging from 30 μM to 0.0015 μM (10 concentrations, 1:3 diluted). After 5 days the CellTiter-Glo™ assay was done according to the instructions of the manufacturer (CellTiter-Glo™ Luminescent Cell Viability Assay, from Promega). In brief: the cell-plate was equilibrated to room temperature for approximately 30 minutes and than the CellTiter-Glo™ reagent was added. The contents were carefully mixed for 15 minutes to induce cell lysis. After 45 minutes the luminescent signal was measured in Victor 2, (scanning multiwell spectrophotometer, Wallac).

### Details:

1st. Day:
Medium: RPMI 1640 with GlutaMAX™ I (Invitrogen, Cat-Nr. 61870), 5% FCS (Sigma Cat.-No. F4135), Pen/Strep (Invitrogen, Cat No. 15140).
HCT116 (ATCC-No. CCl-247): 1000 cells in 60 μl per well of 384 well plate (Greiner 781098, μClear-plate white)
After seeding incubate plates 24 h at 37° C., 5% CO₂
   2nd. Day: Induction (Treatment with Compounds, 10 Concentrations):

In order to achieve a final concentration of 30 μM as highest concentration 3.5 μl of 10 mM compound stock solution were added directly to 163 μl media. Then step e) of the dilution procedure described below, was followed.

In order to achieve the second highest to the lowest concentrations, a serial dilution with dilution steps of 1:3 was followed according to the procedure (a-e) as described here below:
a) for the second highest concentration add 10 μl of 10 mM stock solution of compound to 20 μl dimethylsulfoxide (DMSO)
b) dilute 8×1:3 (always 10 μl to 20 μl DMSO) in this DMSO dilution row (results in 9 wells with concentrations from 3333.3 μM to 0.51 μM)
c) dilute each concentration 1:47.6 (3.5 μl compound dilution to 163 μl media)
e) add 10 μl of every concentration to 60 μl media in the cell plate resulting in final concentration of DMSO: 0.3% in every well and resulting in 10 final concentration of compounds ranging from 30 μM to 0.0015 μM.
   Each compound is tested in triplicate.
   Incubate 120 h (5 days) at 37° C., 5% CO₂

### Analysis:

Add 30 μl CellTiter-Glo™ Reagent (prepared from CellTiter-Glo™ Buffer and CellTiter-Glo™ Substrate (lyophilized) purchased from Promega) per well,
shake 15 minutes at room temperature
incubate further 45 minutes at room temperature without shaking

### Measurement:

Victor 2 scanning multiwell spectrophotometer (Wallac), Luminescence mode (0.5 sec/read, 477 nm)
Determine IC50 using a non-linear curve fit (XLfit software (ID Business Solution Ltd., Guilford, Surrey, UK))

With all compounds a significant inhibition of HCT 116 cell viability was detected, which is exemplified by the compounds shown in Table 2.

TABLE 2
Results:

| Example No. | IC50 HCT 116 [μM] |
|---|---|
| 5 | 0.576 |
| 8 | 0.161 |
| 13 | 0.328 |
| 20 | 0.562 |
| 1, 2, 4, 6, 7, 9, 10, 12, 14, | 0.025-1.500 |
| 16, 18, 19, 21, 22, 24, 25, 26, | |
| 27, 29, 32, 33, 35, 37, 38 | |

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically inert, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

A pharmaceutical compositions comprise e.g. the following:

### a) Tablet Formulation (Wet Granulation):

| Item | Ingredients | Mg/tablet | | | |
|---|---|---|---|---|---|
| 1. | Compound of formula I | 5 | 25 | 100 | 500 |
| 2. | Lactose Anhydrous DTG | 125 | 105 | 30 | 150 |
| | (direct tabletting grade) | | | | |
| 3. | Sta-Rx 1500 (pre- | 6 | 6 | 6 | 30 |
| | gelatinized starch powder) | | | | |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure:

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

### b) Capsule Formulation:

| Item | Ingredients | mg/capsule | | | |
|---|---|---|---|---|---|
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure:

1. Mix items 1, 2 and 3 in a suitable mixer for 30 minutes.
2. Add items 4 and 5 and mix for 3 minutes.
3. Fill into a suitable capsule.

### c) Micro Suspension

1. Weigh 4.0 g glass beads in custom made tube GL 25, 4 cm (the beads fill half of the tube).
2. Add 50 mg compound, disperse with spatulum and vortex.
3. Add 2 ml gelatin solution (weight beads:gelatin solution=2:1) and vortex.
4. Cap and wrap in aluminum foil for light protection.
5. Prepare a counter balance for the mill.
6. Mill for 4 hours, 20/s in a Retsch mill (for some substances up to 24 hours at 30/s).
7. Extract suspension from beads with two layers of filter (100 μm) on a filter holder, coupled to a recipient vial by centrifugation at 400 g for 2 min.
8. Move extract to measuring cylinder.
9. Repeat washing with small volumes(here 1 ml steps) until final volume is reached or extract is clear.
10. Fill up to final volume with gelatin and homogenize.

The following examples are provided to aid the understanding of the present invention, the true scope of which is set forth in the appended claims. It is understood that modifications can be made in the procedures set forth without departing from the spirit of the invention.

### EXPERIMENTAL PROCEDURES

### A: Starting Materials

### Preparation of 5,6-diamino-1-ethyl-3,3-dimethyl-1,3-dihydro-indol-2-one

### i) 1-Ethyl-3,3-dimethyl-6-nitro-1,3-dihydro-indol-2-one

A solution of 3,3-dimethyl-6-nitro-1,3-dihydro-indol-2-one (6 g, 29.10 mmol) in anhydrous N,N-dimethylformamide (DMF) (35 ml) was treated with sodium hydride. The resulting suspension was stirred for 1 h at 60° C. A solution of bromo-ethane (2.17 mL, 3.17 g, 29.10 mmol) in DMF (10 ml) was added. The mixture was allowed to cool to room temperature and stirred for 1 h. After removal of the solvent the mixture was quenched with water (100 ml) and extracted with ethyl acetate (3×100 ml). The extract was dried over Na₂SO₄, evaporated and the crude product was purified by column chromatography on silica gel. Elution with ethyl acetate/n-heptane (1:3) yielded 5.94 g (87%) of a yellow solid.

MS: M=235.3 (ESI+)

¹H-NMR (400 MHz, DMSO): δ (ppm)=1.16 (t, 3H), 1.32 (s, 6H), 3.81 (q, 2H), 7.66 (d, 1H), 7.86 (s, 1H), 7.97 (d, 1H)

### ii) 6-Amino-1-ethyl-3,3-dimethyl-1,3-dihydro-indol-2-one

To a solution of 1-ethyl-3,3-dimethyl-6-nitro-1,3-dihydro-indol-2-one (5.9 g, 25.19 mmol) in methanol/tetrahydrofuran (THF) (1:1, 80 ml) palladium on charcoal (10%, 1.2 g) was added and the mixture hydrogenated at room temperature for 4 h. After filtration and evaporation of the solvents 5.05 g (98%) 6-amino-1-ethyl-3,3-dimethyl-1,3-dihydro-indol-2-one was isolated as white solid.

MS: M=205.0 (API+)

¹H-NMR (400 MHz, DMSO): δ (ppm)=1.11 (t, 3H), 1.17 (s, 6H), 3.58 (q, 2H), 5.12 (br, 2H), 6.21 (d, 1H), 6.25 (s, 1H), 6.92 (d, 1H)

### iii) N-(1-Ethyl-3,3-dimethyl-2-oxo-2,3-dihydro-1H-indol-6-yl)-acetamide

A solution of 6-amino-1-ethyl-3,3-dimethyl-1,3-dihydro-indol-2-one (5.05 g, 24.72 mmol) in acetic anhydride (80 ml) was stirred at room temperature for 4 h. The mixture was poured onto ice water (150 ml), allowed to warm to room temperature and was stirred again for 2 h. After extraction with ethyl acetate (3×100 ml), the combined organic layers were washed with sat. NaHCO₃-solution (3×100 ml), brine (100 ml) and dried over sodium sulfate. After removal of the solvent the crude product was purified by column chromatography on silica gel (ethyl acetate/n-heptane 1:1) yielding 5.6 g (91%) N-(1-ethyl-3,3-dimethyl-2-oxo-2,3-dihydro-1H-indol-6-yl)-acetamide as light yellow solid.

MS: M=247.1 (API+)

¹H-NMR (400 MHz, DMSO): δ (ppm)=1.13 (t, 3H), 1.23 (s, 6H), 2.04 (s, 3H), 3.63 (q, 2H), 7.12 (d, 1H), 7.23 (d, 1H), 7.37 (s, 1H), 9.97 (br, 1H)

### iv) N-(1-ethyl-3,3-dimethyl-5-nitro-2-oxo-2,3-dihydro-1H-indol-6-yl)-acetamide

To a solution of N-(1-ethyl-3,3-dimethyl-2-oxo-2,3-dihydro-1H-indol-6-yl)-acetamide (5.6 g, 22.73 mmol) in acetic anhydride (70 ml) nitric acid (100%, 1.96 g, 1.29 ml, 31.2 mmol) was added at 0° C. The mixture was stirred for 30 min, then poured onto ice water (150 ml). After stirring for 4 h the mixture was extracted with ethyl acetate (3×100 ml). The combined organic layers were washed with sodium hydroxide solution (1M, 100 ml) and water (100 ml), dried over sodium sulfate and concentrated. The crude product was purified by column chromatography on silica gel (ethyl acetate/n-heptane 1:1) to yield 5.2 g (78%) N-(1-ethyl-3,3-dimethyl-5-nitro-2-oxo-2,3-dihydro-1H-indol-6-yl)-acetamide as a yellow solid.

MS: M=292.0 (API+)

¹H-NMR (400 MHz, DMSO): δ (ppm)=1.16 (t, 3H), 1.31 (s, 6H), 2.13 (s, 3H), 3.71 (m, 2H), 7.54 (s, 1H), 8.12 (s, 1H), 10.39 (br, 1H)

### v) 6-Amino-1-ethyl-3,3-dimethyl-5-nitro-1,3-dihydro-indol-2-one

N-(1-ethyl-3,3-dimethyl-5-nitro-2-oxo-2,3-dihydro-1H-indol-6-yl)-acetamide (5.2 g, 17.85 mmol) was dissolved in ethanol (40 ml). After addition of hydrochloric acid (25%, 8 ml, 81.44 mmol) the mixture was stirred under reflux for 3 h. The reaction mixture was allowed to cool down to room temperature and then quenched with water (80 ml). The yellow precipitate was isolated by suction and washed with ethanol/water (1:1). The solid was dissolved in ethyl acetate, dried over sodium sulfate and concentrated to yield 4.15 g (93%) 6-amino-1-ethyl-3,3-dimethyl-5-nitro-1,3-dihydro-indol-2-one as a orange solid.

MS: M=250.0 (API+)

¹H-NMR (400 MHz, DMSO): δ (ppm)=1.15 (t, 3H), 1.27 (s, 6H), 3.64 (m, 2H), 6.54 (s, 1H), 7.67 (br, 2H), 7.95 (s, 1H)

### vi) 5,6-Diamino-1-ethyl-3,3-dimethyl-1,3-dihydro-indol-2-one

To a solution of 6-amino-1-ethyl-3,3-dimethyl-5-nitro-1,3-dihydro-indol-2-one (4.15 g, 16.65 mmol) in ethanol (80 ml) PtO₂ (0.4 g) was added and the mixture hydrogenated at room temperature for 3.5 h. After filtration and evaporation of the solvents 3.25 g (89%) 5,6-diamino-1-ethyl-3,3-dimethyl-1,3-dihydro-indol-2-one was isolated as orange solid.

MS: M=220.0 (API+)

¹H-NMR (400 MHz, DMSO): δ (ppm)=1.10 (t, 3H), 1.13 (s, 6H), 3.53 (m, 2H), 4.08 (br, 2H), 4.48 (br, 2H), 6.27 (s, 1H), 6.50 (s, 1H)

### Preparation of 5,6-Diamino-1-isopropyl-3,3-dimethyl-1,3-dihydro-indol-2-one

5,6-Diamino-1-isopropyl-3,3-dimethyl-1,3-dihydro-indol-2-one was prepared in an analogous 6-step-synthesis as described for 5,6-diamino-1-ethyl-3,3-dimethyl-1,3-dihydro-indol-2-one.

MS: M=234.1 (ESI+)

### Preparation of 5,6-Diamino-3,3-diethyl-1-isopropyl-1,3-dihydro-indol-2-one

### i) 3,3-Diethyl-5-nitro-1,3-dihydro-indol-2-one

To a solution of 3,3-diethyl-1,3-dihydro-indol-2-one (10.0 g, 52.84 mmol, Mertens et al., J. Med. Chem. 30 (1987) 1279-1287) in conc. sulfuric acid (50 ml) was added slowly a mixture of nitric acid (65%, 5.12 g, 3.63 ml, 52.84 mmol) and conc. sulfuric acid (10 ml) at 0° C. After 2 h at room temperature the mixture was poured into ice water. The precipitate was filtered off, washed with water and dried to yield 11.7 g 3,3-diethyl-5-nitro-1,3-dihydro-indol-2-one (49.95 mmol, 94%).

MS: M=235.1 (ESI+)

### ii) 3,3-Diethyl-1-isopropyl-5-nitro-1,3-dihydro-indol-2-one

A solution of 3,3-diethyl-5-nitro-1,3-dihydro-indol-2-one (11.7 g, 49.95 mmol) in anhydrous N,N-dimethylformamide (DMF) (60 ml) was treated with sodium hydride (1.558 g, 64.93 mmol). The resulting suspension was stirred for 1 h at 60° C. A solution of 2-iodo-propane (4.99 ml, 8.49 g, 49.95 mmol) was added. The mixture was kept at 60° C. for further 3 h, allowed to cool to room temperature poured into ice water. The precipitate was filtered off, washed with water and dried to yield 12.6 g 3,3-diethyl-1-isopropyl-5-nitro-1,3-dihydro-indol-2-one (45.60 mmol, 91%).

MS: M=277.1 (ESI+)

### iii) 5-Amino-3,3-diethyl-1-isopropyl-1,3-dihydro-indol-2-one

To a solution of 3,3-diethyl-1-isopropyl-5-nitro-1,3-dihydro-indol-2-one (12.6 g, 45.60 mmol) in methanol/tetrahydrofuran (THF) (1:1, 80 ml) palladium on charcoal (10%, 1.2 g) was added and the mixture hydrogenated at room temperature for 4 h. After filtration of the catalyst the solvent was evaporated and the residue triturated with iso-hexane to yield 9.7 g 5-amino-3,3-diethyl-1-isopropyl-1,3-dihydro-indol-2-one (39.37 mmol, 86%).

MS: M=247.1 (ESI+)

### iv) N-(3,3-Diethyl-1-isopropyl-2-oxo-2,3-dihydro-1H-indol-5-yl)-acetamide

A solution of 5-amino-3,3-diethyl-1-isopropyl-1,3-dihydro-indol-2-one (9.7 g, 39.37 mmol) in acetic anhydride (57 ml) was stirred at room temperature for 4 h. The mixture was poured into ice water, allowed to warm to room temperature and was stirred again for 2 h. After extraction with ethyl acetate, the combined organic layers were washed with aqueous NaOH solution (1M) and brine and dried over sodium sulfate. After removal of the solvent the crude product was triturated with iso-hexane to yield 10.4 g N-(3,3-Diethyl-1-isopropyl-2-oxo-2,3-dihydro-1H-indol-5-yl)-acetamide (36.06 mmol, 91%).

MS: M=289.2 (ESI+)

### v) N-(3,3-Diethyl-1-isopropyl-6-nitro-2-oxo-2,3-dihydro-1H-indol-5-yl)-acetamide

To a solution of N-(3,3-diethyl-1-isopropyl-2-oxo-2,3-dihydro-1H-indol-5-yl)-acetamide (10.4 g, 36.06 mmol) in conc. sulfuric acid (50 ml) was added slowly a mixture of nitric acid (65%, 3.84 g, 2.72 ml, 39.67 mmol) and conc. sulfuric acid (10 ml) at 0° C. After 2 h at room temperature the mixture was poured into ice water. The precipitate was filtered off, washed with water and dried. The crude material was purified by silica gel chromatography (isohexane/ethyl acetate 1:1) to yield 2.2 g N-(3,3-diethyl-1-isopropyl-6-nitro-2-oxo-2,3-dihydro-1H-indol-5-yl)-acetamide (6.60 mmol, 18%) besides undesired N-(3,3-diethyl-1-isopropyl-7-nitro-2-oxo-2,3-dihydro-1H-indol-5-yl)-acetamide (5.5 g).

MS: M=332.2 (ESI−)

### vi) 5-Amino-3,3-diethyl-1-isopropyl-6-nitro-1,3-dihydro-indol-2-one

N-(3,3-diethyl-1-isopropyl-6-nitro-2-oxo-2,3-dihydro-1H-indol-5-yl)-acetamide (2.2 g, 6.60 mmol) was dissolved in ethanol (50 ml). After addition of hydrochloric acid (25%, 3.2 ml, 33.0 mmol) the mixture was heated under reflux for 3 h. Most of the solvent was evaporated and water was added. The mixture was weakly alkalized by addition of aqueous NaOH solution. The mixture was extracted with ethyl acetate, the combined organic phases were dried over magnesium sulfate and the solvent was evaporated to yield 1.9 g 5-amino-3,3-diethyl-1-isopropyl-6-nitro-1,3-dihydro-indol-2-one (6.52 mmol, 99%).

MS: M=290.1 (ESI−)

### vii) 5,6-Diamino-3,3-diethyl-1-isopropyl-1,3-dihydro-indol-2-one

To a solution of 5-amino-3,3-diethyl-1-isopropyl-6-nitro-1,3-dihydro-indol-2-one (1.9 g, 6.52 mmol) in methanol/tetrahydrofuran (THF) (1:1, 80 ml) palladium on charcoal (10%, 1.2 g) was added and the mixture hydrogenated at room temperature for 4 h. After filtration the solvent was evaporated and the residue triturated with iso-hexane to yield 1.7 g 5,6-diamino-3,3-diethyl-1-isopropyl-1,3-dihydro-indol-2-one (6.50 mmol, 99%).

MS: M=262.3 (ESI+)

¹H-NMR (400 MHz, DMSO): δ (ppm)=0.44 (t, 6H), 1.34 (d, 6H), 1.55 (q, 2H), 1.65 (q, 2H), 4.40 (br, 4H), 4.45 (m, 1H), 6.42 (s, 1H), 6.46 (s, 1H)

### Preparation of 5,6-Diamino-1,3,3-triethyl-1,3-dihydro-indol-2-one

5,6-Diamino-1,3,3-triethyl-1,3-dihydro-indol-2-one was prepared in an analogous 7-step-synthesis as described for 5,6-diamino-3,3-diethyl-1-isopropyl-1,3-dihydro-indol-2-one.

MS: M=248.1 (API+)

¹H-NMR (400 MHz, DMSO): δ (ppm)=0.43 (t, 6H), 1.08 (t, 3H), 1.55 (q, 2H), 1.63 (q, 2H), 3.54 (q, 2H), 4.10 (br, 2H), 4.48 (br, 2H), 6.27 (s, 1H), 6.43 (s, 1H)

### Preparation of 3-(5-Ethyl-7,7-dimethyl-6-oxo-3,5,6,7-tetrahydro-imidazo[4,5f]indol-2-yl)-1H-indazole-5-carboxylic acid

### i) 3-Formyl-1H-indazole-5-carboxylic acid

To a mixture of indole-5-carboxylic acid (5.5 g, 0.0338 mol) in water (250 ml) was added NaNO₂ (23.5 g, 0.338 mol) and hydrochloride solution (6N, 42 ml, 0.293 mol). After 12 h at room temperature the precipitate was filtered off, washed with water (270 ml) and dried at 50° C. to yield 5.36 g 3-formyl-1H-indazole-5-carboxylic acid (0.028 mol, 83%) which was used without further purification.

### ii) 3-(5-Ethyl-7,7-dimethyl-6-oxo-3,5,6,7-tetrahydro-imidazo[4,5-f]indol-2-yl)-1H-indazole-5-carboxylic acid

A mixture of 5,6-diamino-1-ethyl-3,3-dimethyl-1,3-dihydro-indol-2-one (1.1 g, 0.005 mol), 3-formyl-1H-indazole-5-carboxylic acid (1.0 g, 0.005 mol) and sulfur (0.176 g, 0.005 mol) in DMF (25 ml) was heated under reflux for 4.5 h. After cooling to room temperature, the reaction mixture was poured into water. After stirring for 15 minutes the precipitate was filtered off, washed thoroughly with water and dried in vacuo over P₂O₅ to yield 1.74 g 3-(5-ethyl-7,7-dimethyl-6-oxo-3,5,6,7-tetrahydro-imidazo[4,5-f]indol-2-yl)-1H-indazole-5-carboxylic acid (0.0044 mol, 89%).

MS: M=390.4 (ESI+)

¹H-NMR (400 MHz, DMSO): δ (ppm)=1.21 (t, 3H), 1.34 (s, 6H), 3.79 (b, 2H), 7.04 and 7.46 (s, 1H, two tautomeric forms), 7.51 and 7.84 (s, 1H, two tautomeric forms), 7.70 (d, 1H), 8.02 (d, 1H), 9.22 and 9.24 (s, 1H, two tautomeric forms), 12.87 (br, 1H), 13.05 and 13.11 (s, 1H, two tautomeric forms), 13.82 and 13.86 (s, 1H, two tautomeric forms)

In an analogous manner as described for 3-(5-ethyl-7,7-dimethyl-6-oxo-3,5,6,7-tetrahydro-imidazo[4,5-f]indol-2-yl)-1H-indazole-5-carboxylic acid the following starting materials were prepared from the appropriate indoles:

| | ¹H-NMR (400 MHz, DMSO): | MS: |
|---|---|---|
| Systematic Name | δ (ppm) = | M = |
| 2-(6-Bromo-1H-indazol- | 1.20 (t, 3H), 1.33 (s, 6H), 3.78 (m, | 425.6 |
| 3-yl)-5-ethyl-7,7- | 2H), 7.03 and 7.37 (s, 1H), 7.44 and | (API+) |
| dimethyl-5,7-dihydro- | 7.72 (s, 1H), 7.45 (m, 1H), 7.89 (m, | |
| 3H-imidazo[4,5-f]indol- | 1H), 8.44 (m, 1H), 13.01 and | |
| 6-one | 13.07 (s, 1H), 13.67 and 13.71 (s, | |
| | 1H) | |
| 2-(5-Bromo-1H-indazol- | 1.21 (m, 3H), 1.33 (s, 6H), 3.78 (m, | 423.9 |
| 3-yl)-5-ethyl-7,7- | 2H), 7.03 and 7.44 (s, 1H), 7.45 and | (ESI−) |
| dimethyl-5,7-dihydro- | 7.78 (s, 1H), 7.58 (m, 1H), 7.65 (m, | |
| 3H-imidazo[4,5-f]indol- | 1H), 8.69 (m, 1H), 13.00 and | |
| 6-one | 13.06 (s, 1H), 13.73 and 13.77 (s, | |
| | 1H) | |
| 3-(5-Ethyl-7,7-dimethyl- | 1.21 (t, 3H), 1.34 (s, 6H), 3.78 (m, | 390.3 |
| 6-oxo-3,5,6,7-tetrahydro- | 2H), 7.04 and 7.40 (s, 1H, two | (ESI+) |
| imidazo[4,5-f]indol-2- | tautomeric forms), 7.46 and 7.74 (s, | |
| yl)-1H-indazole-6- | 1H, two tautomeric forms), 7.87 (d, | |
| carboxylic acid | 1H), 8.23 (s, 1H), 8.57 (d, 1H), | |
| | 13.02 and 13.08 (br, 1H, two | |
| | tautomeric forms), 13.12 (br, 1H), | |
| | 13.86 and 13.90 (br, 1H, two | |
| | tautomeric forms) | |
| 3-(5-Ethyl-7,7-dimethyl- | 1.21 (m, 3H), 1.34 (s, 6H), 3.79 (m, | 371.06 |
| 6-oxo-3,5,6,7-tetrahydro- | 2H), 7.05 and 7.44 (s, 1H), 7.47 and | (ESI+) |
| imidazo[4,5-f]indol-2- | 7.79 (s, 1H), 7.83 (m, 2H), 8.95 (m, | |
| yl)-1H-indazole-5- | 1H), 13.14 and 13.20 (s, 1H), 14.06 | |
| carbonitrile | and 14.09 (s, 1H) | |

### Example 1

### 5-Ethyl-7,7-dimethyl-2-[5-(1H-[1,2,4]triazol-3-yl)-1H-indazol-3-yl]-5,7-dihydro-3H-imidazo[4,5-f]indol-6-one

### i) 3-(5-Ethyl-7,7-dimethyl-6-oxo-3,5,6,7-tetrahydro-imidazo[4,5-f]indol-2-yl)-1H-indazole-5-carboxylic acid amide

To a suspension of 3-(5-ethyl-7,7-dimethyl-6-oxo-3,5,6,7-tetrahydro-imidazo[4,5-f]indol-2-yl)-1H-indazole-5-carboxylic acid (example 69, 500 mg, 1.28 mmol) and DMF (1 drop) in THF (15 ml) at 0° C. under a nitrogen atmosphere was added oxalyl chloride (494 mg, 335 μl, 3.89 mmol). The mixture was allowed to warm to room temperature and stirred for 5.5 h. After 3 and 4 h additional 1 and 0.5 equivalents of oxalyl chloride were added. The reaction mixture was added to an aqueous solution of ammonia (25%, 250 ml, 3339 mmol) stirred for 1 h at room temperature. The aqueous phase was extracted three times with ethyl acetate and the solvent of the combined organic phases was evaporated. The residue was triturated with diisopropyl ether/n-heptane and with water and then dried in vacuum. 410 mg 3-(5-ethyl-7,7-dimethyl-6-oxo-3,5,6,7-tetrahydro-imidazo[4,5-f]indol-2-yl)-1H-indazole-5-carboxylic acid amide (1.056 mmol, 82%) were obtained.

MS: M=389.2 (ESI+)

¹H-NMR (400 MHz, DMSO): δ (ppm)=1.22 (t, 3H), 1.36 (s, 6H), 3.81 (q, 2H), 7.28 (br, 1H), 7.41 (br, 1H), 7.68 (br, 1H), 7.71 (m, 1H), 7.99 (m, 1H), 8.09 (br, 1H), 9.10 (s, 1H), 14.04 (br, 1H)

### ii) 3-(5-Ethyl-7,7-dimethyl-6-oxo-3,5,6,7-tetrahydro-imidazo[4,5-f]indol-2-yl)-1H-indazole-5-carboxylic acid dimethylaminomethyleneamide

A mixture of 3-(5-ethyl-7,7-dimethyl-6-oxo-3,5,6,7-tetrahydro-imidazo[4,5-f]indol-2-yl)-1H-indazole-5-carboxylic acid amide (75 mg, 0.193 mmol) and dimethoxymethyl-dimethyl-amine (336.4 mg, 2.653 mmol) was stirred at 20° C. under a nitrogen atmosphere for 20 minutes. The reaction was quenched with water under ice cooling and the resulting precipitate was filtered off to give 70 mg crude 3-(5-ethyl-7,7-dimethyl-6-oxo-3,5,6,7-tetrahydro-imidazo[4,5-f]indol-2-yl)-1H-indazole-5-carboxylic acid dimethylaminomethyleneamide (70 mg), which was used for the next step without further purification.

### iii) 5-Ethyl-7,7-dimethyl-2-[5-(1H-[1,2,4]triazol-3-yl)-1H-indazol-3-yl]-5,7-dihydro-3H-imidazo[4,5-f]indol-6-one

A mixture of 3-(5-ethyl-7,7-dimethyl-6-oxo-3,5,6,7-tetrahydro-imidazo[4,5-f]indol-2-yl)-1H-indazole-5-carboxylic acid dimethylaminomethyleneamide (70 mg, crude), hydrazone hydrate (41.3 mg, 0.825 mmol) and glacial acetic acid (350 μl) was heated at 75° C. for one hour and then cooled to room temperature. Water was added and the aqueous phase was extracted three times with ethyl acetate. The combined organic phases were dried over MgSO₄ the solvent was evaporated. The residue was triturated with diethyl ether and purified by silica gel chromatography (dichloromethane/methanol 9:1) to yield 41 mg 5-ethyl-7,7-dimethyl-2-[5-(1H-[1,2,4]triazol-3-yl)-1H-indazol-3-yl]-5,7-dihydro-3H-imidazo[4,5-f]indol-6-one (0.0994 mmol, 63%)

MS: M=413.18 (ESI+)

¹H-NMR (400 MHz, DMSO): δ (ppm)=14.58-13.51 (bm, 2H), 13.01 (m, 1H), 9.22 (s, 1H), 8.49 (s, 1H), 8.14 (d, 1H), 7.84 and 7.51 (s, 1H), 7.73 (d, 1H), 7.46 and 7.04 (s, 1H), 3.79 (m, 2H), 1.34 (s, 6H), 1.23 (m, 3H)

### Example 2

### 5-Ethyl-7,7-dimethyl-2-[6-(1H-[1,2,4]triazol-3-yl)-1H-indazol-3-yl]-5,7-dihydro-3H-imidazo[4,5-f]indol-6-one

In an analogous manner as described for example 1 5-ethyl-7,7-dimethyl-2-[6-(1H-[1,2,4]triazol-3-yl)-1H-indazol-3-yl]-5,7-dihydro-3H-imidazo[4,5-f]indol-6-one was prepared from 3-(5-ethyl-7,7-dimethyl-6-oxo-3,5,6,7-tetrahydro-imidazo[4,5-f]indol-2-yl)-1H-indazole-6-carboxylic acid.

MS: M=413.3 (ESI+)

¹H-NMR (400 MHz, DMSO): δ (ppm)=13.71 (m, 2H); 13.01 (m, 1H); 8.58-8.52 (bm, 2H); 8.27 (s, 1H); 8.02 (d, 1H); 7.75 and 7.46 (s, 1H); 7.40 and 7.04 (s, 1H); 1.35 (s, 6H); 1.22 (t, 3H)

### Example 3

### 5-Ethyl-7,7-dimethyl-2-[5-(1H-tetrazol-5-yl)-1H-indazol-3-yl]-5,7-dihydro-3H-imidazo[4,5-f]indol-6-one

A mixture of 3-(5-ethyl-7,7-dimethyl-6-oxo-3,5,6,7-tetrahydro-imidazo[4,5-f]indol-2-yl)-1H-indazole-5-carbonitrile (55 mg, 0.15 mmol), trimethyltin azide (123 mg, 0.6 mmol) and DMF (4 ml) is heated to 150° C. for 3 days. The reaction mixture was cooled to room temperature, treated with water and evaporated to dryness. The residue was treated three times with ethanol followed by evaporation of the solvent. The residue was triturated with ethyl acetate to yield 5-ethyl-7,7-dimethyl-2-[5-(1H-tetrazol-5-yl)-1H-indazol-3-yl]-5,7-dihydro-3H-imidazo[4,5-f]indol-6-one (58 mg, 0.14 mmol, 93%)

MS: M=414.15 (ESI+)

¹H-NMR (400 MHz, DMSO): δ (ppm)=13.97 (m, 1H), 9.28 (s, 1H), 8.12 (d, 1H), 7.88 (d, 1H), 7.67 (m, 1H), 7.25 (m, 1H), 3.80 (q, 2H), 1.35 (s, 6H), 1.22 (t, 3H)

### Example 4

### 5-Ethyl-7,7-dimethyl-2-(6-thiophen-3-yl-1H-indazol-3-yl)-5,7-dihydro-3H-imidazo[4,5-f]indol-6-one

### i) 2-[6-Bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one

A solution of 2-(6-bromo-1H-indazol-3-yl)-5-ethyl-7,7-dimethyl-5,7-dihydro-3H-imidazo[4,5-f]indol-6-one (860 mg, 2.027 mmol) in THF (15 ml) at 0° C. under an argon atmosphere was treated with sodium tert-butoxide (430 mg, 4.474 mmol). After one hour at 0° C. (2-chloromethoxy-ethyl)-trimethyl-silane (1017.4 mg, 6.102 mmol) was added. After 2 h two further equivalents (2-chloromethoxy-ethyl)-trimethyl-silane were added and the reaction mixture was allowed to warm to room temperature. After 1.5 h the reaction mixture was treated with water and the aqueous phase was extracted with ethyl acetate. The combined organic phases were dried over MgSO₄ and the solvent was evaporated. The residue was purified by silica gel chromatography (ethyl acetate) to yield crude 2-[6-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one (1798 mg) which was used for the next step.

### ii) 5-Ethyl-7,7-dimethyl-2-[6-thiophen-3-yl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one

To a solution of 2-[6-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one (120 mg, 0.175 mmol) in toluene (2 ml) and methanol (0.3 ml) under an argon atmosphere were added tetrakis(triphenylphosphin)palladium (20.2 mg, 0.017 mmol), thiophene-3-boronic acid (33.6 mg, 0.263 mmol) and saturated aqueous sodium bicarbonate solution (480 μl). After heating to 90° C. for 5.5 h the reaction mixture was allowed to cool to room temperature and was treated with water. The aqueous phase was extracted three times with ethyl acetate. The combined organic phases were dried over MgSO₄ and the solvent was evaporated. The residue was purified by HPL chromatography to yield 5-ethyl-7,7-dimethyl-2-[6-thiophen-3-yl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (61.3 mg, 0.089 mmol, 51%).

### iii) 5-Ethyl-7,7-dimethyl-2-(6-thiophen-3-yl-1H-indazol-3-yl)-5,7-dihydro-3H-imidazo[4,5-f]indol-6-one

A mixture of 5-ethyl-7,7-dimethyl-2-[6-thiophen-3-yl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (61.3 mg, 0.089 mmol), tetra-n-butylammonium fluoride (1M solution THF, 1.834 ml) and ethylenediamine (54.4 mg, 0.905 mmol) was heated at 70° C. for 48 h. The reaction mixture was allowed to cool to room temperature and was treated with water. The aqueous phase was extracted three times with ethyl acetate. The combined organic phases were dried over MgSO₄ and the solvent was evaporated. The residue was purified by HPL chromatography to yield 5-ethyl-7,7-dimethyl-2-(6-thiophen-3-yl-1H-indazol-3-yl)-5,7-dihydro-3H-imidazo[4,5-f]indol-6-one (27.8 mg, 0.065 mmol, 73%).

MS: M=426.2 (ESI−)

¹H-NMR (400 MHz, DMSO): δ (ppm)=1.22 (t, 3H), 1.35 (s, 6H), 3.80 (m, 2H), 7.04 and 7.74 (s, 1H, two tautomeric forms), 7.42 (d, 1H), 7.70 (m, 3H), 7.89 (s, 1H), 8.03 (m, 1H), 8.50 (m, 1H), 12.96 (m, 1H), 13.58 (s, 1H)

In an analogous manner as described for example 4 the following examples 5-23 were prepared from 2-(6-bromo-1H-indazol-3-yl)-5-ethyl-7,7-dimethyl-5,7-dihydro-3H-imidazo[4,5-f]indol-6-one and the appropriate boronic acids respectively boronic acid esters:

| Example | | 1H-NMR (400 MHz, | |
|---|---|---|---|
| No. | Systematic Name | DMSO): δ (ppm) = | MS: M = |
| 5 | 5-Ethyl-7,7-dimethyl-2- | 13.60 (m, 1H), 12.97 (m, | 448.27 (ESI+) |
| | [6-((E)-styryl)-1H- | 1H), 8.46 (m, 1H), | |
| | indazol-3-yl]-5,7- | 7.80-7.00 (bm, 11H), 3.78 (m, | |
| | dihydro-3H- | 2H), 1.34 (s, 6H), 1.21 (m, | |
| | imidazo[4,5-f]indol-6- | 3H) | |
| | one | | |
| 6 | 5-Ethyl-2-{6-[(E)-2-(4- | 13.59 (m, 1H), 12.96 (m, | 466.17 (ESI+) |
| | fluoro-phenyl)-vinyl]- | 1H), 8.46 (m, 1H), | |
| | 1H-indazol-3-yl}-7,7- | 7.78-7.00 (bm, 10H), 3.79 (m, | |
| | dimethyl-5,7-dihydro- | 2H), 1.34 (s, 6H), 1.21 (t, | |
| | 3H-imidazo[4,5-f]indol- | 3H) | |
| | 6-one | | |
| 7 | 5-Ethyl-7,7-dimethyl-2- | 13.48 (m, 1H), 12.94 (m, | 426.17 (ESI+) |
| | [6-(1-methyl-1H- | 1H), 8.44 (m, 1H), 8.28 (s, | |
| | pyrazol-4-yl)-1H- | 1H), 7.99 (s, 1H), 7.73 (m, | |
| | indazol-3-yl]-5,7- | 1H), 7.73 and 7.44 (s, 1H), | |
| | dihydro-3H- | 7.54 (m, 1H), 7.38 and | |
| | imidazo[4,5-f]indol-6- | 7.03 (s, 1H), 3.90 (s, 3H), | |
| | one | 3.79 (m, 2H), 1.34 (s, 6H), | |
| | | 1.21 (t, 3H) | |
| 8 | 5-Ethyl-7,7-dimethyl-2- | 13.74 (m, 1H), 13.01 (m, | 421.03 (ESI−) |
| | (6-pyridin-3-yl-1H- | 1H), 9.02 (m, 1H), | |
| | indazol-3-yl)-5,7- | 8.70-8.57 (m, 2H), 8.21 (m, | |
| | dihydro-3H- | 1H), 7.93 (s, 1H), 7.73 and | |
| | imidazo[4,5-f]indol-6- | 7.47 (s, 1H), 7.67 (m, 1H), | |
| | one | 7.55 (m, 1H), 7.40 and | |
| | | 7.05 (s, 1H), 3.79 (q, 2H), | |
| | | 1.34 (s, 6H), 1.22 (t, 3H) | |
| 9 | 2-[6-((E)-2-Biphenyl-4- | 13.60 (m, 1H), 12.97 (m, | 524.15 (ESI+) |
| | yl-vinyl)-1H-indazol-3- | 1H), 8.48 (m, 1H), | |
| | yl]-5-ethyl-7,7- | 7.80-7.02 (bm, 15H), 3.79 (m, | |
| | dimethyl-5,7-dihydro- | 2H), 1.34 (m, 6H), | |
| | 3H-imidazo[4,5-f]indol- | 1.22 (m, 3H) | |
| | 6-one | | |
| 10 | 5-Ethyl-2-{6-[(E)-2-(4- | 13.55 (m, 1H), 12.95 (m, | 478.39 (ESI+) |
| | methoxy-phenyl)- | 1H), 8.44 (m, 1H), | |
| | vinyl]-1H-indazol-3-yl}- | 7.78-6.95 (bm, 10H), | |
| | 7,7-dimethyl-5,7- | 3.85-3.73 (m, 5H), 1.34 (m, 6H), | |
| | dihydro-3H- | 1.21 (t, 3H) | |
| | imidazo[4,5-f]indol-6- | | |
| | one | | |
| 11 | 5-Ethyl-7,7-dimethyl-2- | 13.65 (m, 1H), 12.98 (m, | 516.18 (ESI+) |
| | {6-[(E)-2-(4- | 1H), 8.49 (m, 1H), | |
| | trifluoromethyl- | 7.94-6.99 (bm, 10H), 3.79 (m, | |
| | phenyl)-vinyl]-1H- | 2H), 1.34 (m, 6H), 1.22 (t, | |
| | indazol-3-yl}-5,7- | 3H) | |
| | dihydro-3H- | | |
| | imidazo[4,5-f]indol-6- | | |
| | one | | |
| 12 | 2-[6-(4- | 13.48 (m, 1H), 12.94 (m, | 465.34 (ESI+) |
| | Dimethylamino- | 1H), 8.48 (m, 1H), 7.73 | |
| | phenyl)-1H-indazol-3- | and 7.44 (s, 1H), | |
| | yl]-5-ethyl-7,7- | 7.72-7.53 (m, 4H), 7.39 and | |
| | dimethyl-5,7-dihydro- | 7.03 (s, 1H), 6.85 (m, 2H), | |
| | 3H-imidazo[4,5-f]indol- | 3.79 (m, 2H), 2.97 (s, 6H), | |
| | 6-one | 1.34 (m, 6H), 1.22 (m, 3H) | |
| 13 | 2-[6-(4-Acetyl-phenyl)- | 1.22 (t, 3H), 1.34 (s, 6H), | 462.3 (ESI−) |
| | 1H-indazol-3-yl]-5- | 2.64 (s, 3H), 3.79 (m, 2H), | |
| | ethyl-7,7-dimethyl-5,7- | 7.05 and 7.75 (s, 1H, two | |
| | dihydro-3H- | tautomeric forms), 7.43 (d, | |
| | imidazo[4,5-f]indol-6- | 1H), 7.69 (d, 1H), 7.95 (m, | |
| | one | 3H), 8.10 (m, 2H), 8.60 (t, | |
| | | 1H), 13.00 (m, 1H), | |
| | | 13.71 (s, 1H) | |
| 14 | 5-Ethyl-2-[6-(6- | 1.22 (t, 3H), 1.34 (s, 6H), | 451.2 (ESI−) |
| | methoxy-pyridin-3-yl)- | 3.79 (m, 2H), 3.93 (s, 3H), | |
| | 1H-indazol-3-yl]-7,7- | 6.97 (d, 1H), 7.04 and | |
| | dimethyl-5,7-dihydro- | 7.74 (s, 1H, two tautomeric | |
| | 3H-imidazo[4,5-f]indol- | forms), 7.42 (d, 1H), | |
| | 6-one | 7.61 (d, 1H), 7.83 (s, 1H), | |
| | | 8.14 (m, 1H), 8.55 (d, 1H), | |
| | | 8.61 (d, 1H), 12.98 (m, 1H), | |
| | | 13.65 (s,1H) | |
| 15 | 5-Ethyl-7,7-dimethyl-2- | 1.22 (t, 3H), 1.35 (s, 6H), | 421.2 (ESI−) |
| | (6-pyridin-4-yl-1H- | 3.80 (m, 2H), 7.05 and | |
| | indazol-3-yl)-5,7- | 7.72 (s, 1H, two | |
| | dihydro-3H- | tautomeric forms), | |
| | imidazo[4,5-f]indol-6- | 7.43 (m, 1H), 7.75 (s, 1H), | |
| | one | 7.84-7.85 (m, 2H), 8.02 (s, | |
| | | 1H), 8.62 (d, 1H), 8.70 (d, | |
| | | 2H), 13.04 (d, 1H), | |
| | | 13.78 (s, 1H) | |
| 16 | 5-Ethyl-7,7-dimethyl-2- | 1.22 (t, 3H), 1.35 (s, 6H), | 428.3 (ESI+) |
| | (6-thiophen-2-yl-1H- | 3.80 (m, 2H), 7.05 and | |
| | indazol-3-yl)-5,7- | 7.62 (s, 1H, two | |
| | dihydro-3H- | tautomeric forms), | |
| | imidazo[4,5-f]indol-6- | 7.21 (m, 1H), 7.40 and 7.46 (s, | |
| | one | 1H, two tautomeric | |
| | | forms), 7.63-7.68 (m, | |
| | | 2H), 7.75 and 7.67 (s, 1H, | |
| | | two tautomeric forms), | |
| | | 7.84 (s, 1H), 8.51 (m, 1H), | |
| | | 12.98 (d, 1H), 13.60 (s, | |
| | | 1H) | |
| 17 | 4-[3-(5-Ethyl-7,7- | 1.22 (t, 3H), 1.34 (s, 6H), | 466.1 (ESI+) |
| | dimethyl-6-oxo-3,5,6,7- | 3.79 (m, 2H), 7.04 and | |
| | tetrahydro-imidazo[4,5- | 7.75 (s, 1H, two | |
| | f]indol-2-yl)-1H- | tautomeric forms), 7.43 (d, | |
| | indazol-6-yl]-benzoic | 1H), 7.69 (m, 1H), | |
| | acid | 7.92 (m, 3H), 8.07 (d, 2H), | |
| | | 8.59 (t, 1H), 13.01 (d, 1H), | |
| | | 13.72 (d, 1H) | |
| 18 | 2-{6-[(E)-2-(4-Chloro- | 1.21 (t, 3H), 1.34 (s, 6H), | 482.1 (ESI+) |
| | phenyl)-vinyl]-1H- | 3.79 (m, 2H), 7.03 and | |
| | indazol-3-yl}-5-ethyl- | 7.39 (s, 1H, two | |
| | 7,7-dimethyl-5,7- | tautomeric forms), | |
| | dihydro-3H- | 7.43-7.75 (m, 9H), 8.47 (m, | |
| | imidazo[4,5-f]indol-6- | 1H), 12.97 (d, 1H), | |
| | one | 13.61 (s, 1H) | |
| 19 | 2-[6-((E)-2-Cyclohexyl- | 1.17 (m, 3H), 1.33 (m, | 454.2 (ESI+) |
| | vinyl)-1H-indazol-3-yl]- | 6H), 1.66 (d, 1H), 1.78 (m, | |
| | 5-ethyl-7,7-dimethyl- | 4H), 1.91 (s, 1H), 3.78 (m, | |
| | 5,7-dihydro-3H- | 2H), 4.02 (m, 4H), | |
| | imidazo[4,5-f]indol-6- | 6.40 (m, 1H), 6.56 (d, 1H), 7.02 | |
| | one | and 7.38 (s, 1H, two | |
| | | tautomeric forms), 7.43 (s, | |
| | | 1H), 7.44 and 7.72 (s, 1H, | |
| | | two tautomeric forms), | |
| | | 7.50 (s, 1H), 8.38 (m, 1H), | |
| | | 12.92 (d, 1H), 13.46 (d, | |
| | | 1H) | |
| 20 | 2-(6-Benzo[1,3]dioxol- | 1.17 (t, 3H), 1.28 (s, 6H), | 464.3 (ESI−) |
| | 5-yl-1H-indazol-3-yl)- | 3.73 (m, 2H), 5.99 (s, 2H), | |
| | 5-ethyl-7,7-dimethyl- | 6.93 (d, 1H), 7.14 (s, 1H), | |
| | 5,7-dihydro-3H- | 7.18 (m, 1H), 7.24 (d, 1H), | |
| | imidazo[4,5-f]indol-6- | 7.48 (d, 1H), 7.54 (s, 1H), | |
| | one | 7.69 (s, 1H), 8.44 (d, 1H), | |
| 21 | 2-[6-(3- | 1.22 (t, 3H), 1.33 (s, 6H), | 463.3 (ESI−) |
| | Dimethylamino- | 1.88 (s, 6H), 3.78 (m, 2H), | |
| | phenyl)-1H-indazol-3- | 6.79 (d, 1H), 7.04 (s, 2H), | |
| | yl]-5-ethyl-7,7- | 7.31 (m, 1H), 7.60 (d, 1H), | |
| | dimethyl-5,7-dihydro- | 7.80 (s, 1H), 8.52 (d, 1H), | |
| | 3H-imidazo[4,5-f]indol- | | |
| | 6-one | | |
| 22 | 5-Ethyl-7,7-dimethyl-2- | 1.22 (t, 3H), 1.34 (s, 6H), | 465.3 (ESI−) |
| | [6-(3-nitro-phenyl)-1H- | 3.79 (m, 2H), 7.72 (d, 1H), | |
| | indazol-3-yl]-5,7- | 7.82 (t, 1H), 8.00 (s, 1H), | |
| | dihydro-3H- | 8.28 (m, 2H), 8.56 (s, 1H), | |
| | imidazo[4,5-f]indol-6- | 8.62 (d, 1H) | |
| | one | | |
| 23 | 5-Ethyl-2-{6-[(E)-2-(3- | 1.21 (m, 3H), 2.50 (s, 6H), | 464.3 (ESI−) |
| | fluoro-phenyl)-vinyl]- | 3.79 (m, 2H), | |
| | 1H-indazol-3-yl}-7,7- | 7.03-8.48 (m, 11H), 12.97 (d, 1H), | |
| | dimethyl-5,7-dihydro- | 13.63 (d, 1H) | |
| | 3H-imidazo[4,5-f]indol- | | |
| | 6-one | | |

In an analogous manner as described for example 4 the following examples 24-30 were prepared from 2-(5-bromo-1H-indazol-3-yl)-5-ethyl-7,7-dimethyl-5,7-dihydro-3H-imidazo[4,5-f]indol-6-one and the appropriate boronic acids respectively boronic acid esters:

| Example | | 1H-NMR (400 MHz, | |
|---|---|---|---|
| No. | Systematic Name | DMSO): d (ppm) = | MS: M = |
| 24 | 5-Ethyl-2-[5-(6- | 13.64 (m, 1H); | 453.3 (ESI+) |
| | methoxy-pyridin-3-yl)- | 12.99 (m, 1H); 8.67 (d, 1H); | |
| | 1H-indazol-3-yl]-7,7- | 8.5296 (s, 1H); 8.07 (d, 1H); | |
| | dimethyl-5,7-dihydro- | 7.78 and 7.03 (s, 1H); | |
| | 3H-imidazo[4,5- | 7.75 (m, 2H); 7.44 (s, 1H); | |
| | f]indol-6-one; | 6.98 (m, 1H); 3.93 (s, 3H); | |
| | compound with acetic | 3.78 (m, 2H); 1.33 (s, 6H); | |
| | acid | 1.20 (t, 3H) | |
| 25 | 5-Ethyl-7,7-dimethyl-2- | 13.61 (m, 1H); | 428.3 (ESI+) |
| | (5-thiophen-3-yl-1H- | 12.97 (m, 1H); 8.73 (d, 1H); | |
| | indazol-3-yl)-5,7- | 7.86 (s, 1H); 7.84 and | |
| | dihydro-3H- | 7.82 (s, 1H); 7.77 and 7.03 (s, | |
| | imidazo[4,5-f]indol-6- | 1H); 7.72-7.66 (bm, 2H), | |
| | one; compound with | 7.59 (d, 1H); 7.43 (d, 1H); | |
| | acetic acid | 3.78 (m, 2H); 1.33 (s, 6H); | |
| | | 1.21 (t, 3H) | |
| 26 | 5-Ethyl-7,7-dimethyl-2- | 13.53 (s, 1H); | 426.3 (ESI+) |
| | [5-(1-methyl-1H- | 12.94 (m, 1H); 8.57 (s, 1H); | |
| | pyrazol-4-yl)-1H- | 8.18 (s, 1H); 7.87 (s, 1H); 7.75 | |
| | indazol-3-yl]-5,7- | and 7.03 (s, 1H); | |
| | dihydro-3H- | 7.69-7.62 (bm, 2H); 7.44 and | |
| | imidazo[4,5-f]indol-6- | 7.40 (s, 1H); 3.91 (s, 3H); | |
| | one; compound with | 3.78 (m, 2H); 1.33 (s, 6H); | |
| | acetic acid | 1.21 (t, 3H) | |
| 27 | 5-Ethyl-7,7-dimethyl-2- | 13.70 (s, 1H); | 423.3 (ESI+) |
| | (5-pyridin-3-yl-1H- | 13.02 (m, 1H); 8.96 (d, 1H); | |
| | indazol-3-yl)-5,7- | 8.76 (s, 1H); 8.61 (d, 1H); | |
| | dihydro-3H- | 8.15 (d, 1H); 7.84 and | |
| | imidazo[4,5-f]indol-6- | 7.04 (s, 1H); 7.82-7.77 (bm, 2H); | |
| | one | 7.55 (m, 1H); 7.44 (d, 1H); | |
| | | 3.78 (d, 2H); 1.33 (s, 6H), | |
| | | 1.20 (t, 3H) | |
| 28 | 2-[5-(4- | 13.49 (m, 1H); | 465.3 (ESI+) |
| | Dimethylamino- | 12.90 (m, 1H); 8.63 (d, 1H); 7.77 | |
| | phenyl)-1H-indazol-3- | and 7.03 (s, 1H); | |
| | yl]-5-ethyl-7,7- | 7.71 (d, 1H); 7.66 (m, 1H); | |
| | dimethyl-5,7-dihydro- | 7.58 (d, 2H); 7.43 (d, 1H); | |
| | 3H-imidazo[4,5- | 6.88 (d, 2H); 3.78 (m, 2H); | |
| | f]indol-6-one | 2.97 (s, 6H); 1.34 (d, 6H); | |
| | | 1.21 (m, 3H) | |
| 29 | 2-[5-(3- | 13.59 (m, 1H); | 465.3 (ESI+) |
| | Dimethylamino- | 12.97 (m, 1H); 8.68 (s, 1H); | |
| | phenyl)-1H-indazol-3- | 7.77 (m, 1H); 7.74 and | |
| | yl]-5-ethyl-7,7- | 7.03 (s, 1H); 7.70 (m, 1H); | |
| | dimethyl-5,7-dihydro- | 7.43 (d, 1H); 7.32 (t, 1H); | |
| | 3H-imidazo[4,5- | 6.99 (d, 2H); 6.77 (d, 1H); | |
| | f]indol-6-one | 3.78 (m, 2H); 2.99 (s, 6H); | |
| | | 1.33 (s, 6H); 1.20 (t, 3H) | |
| 30 | 2-(5-Benzo[1,3]dioxol- | 13.60 (m, 1H); | 466.3 (ESI+) |
| | 5-yl-1H-indazol-3-yl)- | 12.97 (m, 1H); 8.64 (s, 1H); 7.79 | |
| | 5-ethyl-7,7-dimethyl- | and 7.03 (s, 1H); | |
| | 5,7-dihydro-3H- | 7.70 (m, 2H); 7.44 (s, 1H); | |
| | imidazo[4,5-f]indol-6- | 7.28 (d, 1H); 7.20 (d, 1H); | |
| | one; compound with | 7.08 and 7.06 (s, 1H); | |
| | acetic acid | 6.10 (s, 2H); 3.78 (m, 2H); | |
| | | 1.33 (s, 6H); 1.20 (t, 3H) | |

### Example 31

### 5-Ethyl-7,7-dimethyl-2-(6-phenyl-1H-indazol-3-yl)-5,7-dihydro-3H-imidazo[4,5-f]indol-6-one

### i) 5-Ethyl-7,7-dimethyl-2-[6-(4,4,5,5-tetramethyl-[1,3,2]dioxaborolan-2-yl)-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one

To a solution of 2-[6-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one (see example 41, 400 mg, 0.584 mmol) in DMF (2 ml) under an argon atmosphere were added bis(pinacolato) diboron (164.6 mg, 0.648 mmol), potassium acetate (172 mg, 1.752 mmol) and 1,1′-bis(diphenylphosphino)ferrocene palladium (II) chloride dichloromethane adduct (23.8 mg, 0.029 mmol). After heating to 75° C. for 14 h the reaction mixture was allowed to cool to room temperature and was purified by silica gel chromatography (ethyl acetate) to yield 5-ethyl-7,7-dimethyl-2-[6-(4,4,5,5-tetramethyl-[1,3,2]dioxaborolan-2-yl)-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (413 mg, 0.564 mmol, 97%).

### ii) 5-Ethyl-7,7-dimethyl-2-[6-phenyl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one

To a solution of 5-ethyl-7,7-dimethyl-2-[6-(4,4,5,5-tetramethyl-[1,3,2]dioxaborolan-2-yl)-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (106.9 mg, 0.146 mmol) in toluene (2 ml) and methanol (0.3 ml) under an argon atmosphere were added bromo-benzene (35.8 mg, 0.228 mmol), tetrakis(triphenylphosphin)palladium (17 mg, 0.015 mmol) and saturated aqueous sodium bicarbonate solution (400 μl). After heating to 90° C. for 6.5 h the reaction mixture was allowed to cool to room temperature and was treated with water. The aqueous phase was extracted three times with ethyl acetate. The combined organic phases were dried over MgSO₄ and the solvent was evaporated. The residue was purified by HPL chromatography to yield 5-ethyl-7,7-dimethyl-2-[6-phenyl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (39.5 mg, 0.058 mmol, 40%).

### iii) 5-Ethyl-7,7-dimethyl-2-(6-phenyl-1H-indazol-3-yl)-5,7-dihydro-3H-imidazo[4,5-f]indol-6-one

A mixture of 5-ethyl-7,7-dimethyl-2-[6-phenyl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (39.5 mg, 0.058 mmol), tetra-n-butylammonium fluoride (1M solution THF, 1.15 ml) and ethylenediamine (35 mg, 0.582 mmol) was heated at 70° C. for 48 h. The reaction mixture was allowed to cool to room temperature and was treated with water. The aqueous phase was extracted three times with ethyl acetate. The combined organic phases were dried over MgSO₄ and the solvent was evaporated. The residue was purified by HPL chromatography to yield 5-ethyl-7,7-dimethyl-2-(6-phenyl-1H-indazol-3-yl)-5,7-dihydro-3H-imidazo[4,5-f]indol-6-one (27.8 mg, 0.065 mmol, 73%).

In an analogous manner as described for example 31 the following examples 32-34 were prepared from 2-[6-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one and the appropriate aryl bromides:

| | | 1H-NMR (400 MHz, | |
|---|---|---|---|
| Example No. | Systematic Name | DMSO): d (ppm) = | MS: M = |
| 32 | 5-Ethyl-7,7-dimethyl-2- | 1.22 (m, 3H), 1.34 (s, 6H), | 422.2 (ESI−) |
| | (6-pyrimidin-5-yl-1H- | 3.79 (m, 2H), 7.04 and | |
| | indazol-3-yl)-5,7- | 7.73 (s, 1H, two | |
| | dihydro-3H- | tautomeric forms), 7.44 (d, | |
| | imidazo[4,5-f]indol-6- | 1H), 7.75 (s, 1H), 8.05 (s, | |
| | one | 1H), 8.64 (m, 1H), | |
| | | 9.27 (m, 3H), 13.04 (d, 1H), | |
| | | 13.82 (s, 1H) | |
| 33 | 5-Ethyl-7,7-dimethyl-2- | 1.22 (t, 3H), 1.34 (s, 6H), | 421.3 (ESI−) |
| | (6-pyridin-2-yl-1H- | 3.79 (m, 2H), 7.05 and | |
| | indazol-3-yl)-5,7- | 7.74 (s, 1H, two | |
| | dihydro-3H- | tautomeric forms), | |
| | imidazo[4,5-f]indol-6- | 7.41 (m, 2H), 7.94 (m, 1H), | |
| | one | 8.06-8.13 (m, 2H), | |
| | | 8.33 (s, 1H), 8.58 (d, 1H), | |
| | | 8.73 (d, 1H) | |
| 34 | 2-[6-(3,5-Dimethoxy- | 1.16 (t, 3H), 1.28 (s, 6H), | 480.3 (ESI−) |
| | phenyl)-1H-indazol-3- | 3.76 (s, 6H), 6.46 and | |
| | yl]-5-ethyl-7,7- | 6.81 (s, 1H, two tautomeric | |
| | dimethyl-5,7-dihydro- | forms), 6.80 (s, 1H), | |
| | 3H-imidazo[4,5- | 7.15 (s, 1H), 7.32 and 7.53 (m, | |
| | f]indol-6-one | 1H), 7.42 (t, 1H), 7.55 (m, | |
| | | 2H), 7.68 (m, 1H), 7.77 8s, | |
| | | 1H), 8.46 (m, 1H) | |

In an analogous manner as described for example 31 the following examples 32-34 were prepared from 2-[5-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one and the appropriate aryl bromides:

| Ex- | | | |
|---|---|---|---|
| am- | | | |
| ple | | 1H-NMR (400 MHz, | MS: |
| No. | Systematic Name | DMSO): d (ppm) = | M = |
| 35 | 5-Ethyl-7,7-dimethyl-2- | 13.76 (s, 1H), 13.03 (s, 1H); | 424.3 |
| | (5-pyrimidin-5-yl-1H- | 9.23 (s, 1H); 9.20 (s, 1H); | (ESI+) |
| | indazol-3-yl)-5,7- | 8.80 (s, 1H); 7.88 (d, 1H); | |
| | dihydro-3H- | 7.81 (d, 1H); 7.77 and | |
| | imidazo[4,5-f]indol-6- | 7.04 (s, 1H); 7.44 (s, 1H), | |
| | one | 3.78 (m, 2H); 1.33 (s, 6H); | |
| | | 1.20 (t, 3H) | |
| 36 | 5-Ethyl-7,7-dimethyl-2- | 13.73 (s, 1H); 13.03 (s, 1H); | 423.3 |
| | (5-pyridin-2-yl-1H- | 9.24 (s, 1H); 8.73 (d, 1H); | (ESI+) |
| | indazol-3-yl)-5,7- | 8.20 (d, 1H); 8.03 (d, 1H); | |
| | dihydro-3H- | 7.93 (t, 1H); 7.82 and | |
| | imidazo[4,5-f]indol-6- | 7.05 (s, 1H); 7.73 (d, 1H); | |
| | one | 7.47 (s; 1H); 7.37 (t, 1H); | |
| | | 3.79 (m, 2H); 1.34 (s, 6H); | |
| | | 1.21 (t, 3H) | |

### Example 37

### 5-Ethyl-7,7-dimethyl-2-[6-(1H-pyrazol-4-yl)-1H-indazol-3-yl]-5,7-dihydro-3H-imidazo[4,5-f]indol-6-one

### i) 4-Iodo-1-(2-trimethylsilanyl-ethoxymethyl)-1H-pyrazole

A solution of 4-iodo-1H-pyrazole (1000 mg, 5.104 mmol) in THF (20 ml) at 0° C. under a nitrogen atmosphere was treated with sodium tert-butoxide (1079 mg, 11.23 mmol). After one hour at room temperature (2-chloromethoxy-ethyl)-trimethyl-silane (2253 mg, 15.31 mmol) was added. After 48 h at room temperature the reaction mixture was treated with water and the aqueous phase was extracted with ethyl acetate. The combined organic phases were dried over MgSO₄ and the solvent was evaporated. The residue was purified by HPL chromatography to yield 4-iodo-1-(2-trimethylsilanyl-ethoxymethyl)-1H-pyrazole (1050 mg, 3.24 mmol, 63%).

### ii) 5-Ethyl-7,7-dimethyl-2-[6-(1H-pyrazol-4-yl)-1H-indazol-3-yl]-5,7-dihydro-3H-imidazo[4,5-f]indol-6-one

In an analogous manner as described for example 32 ii) and iii) 5-ethyl-7,7-dimethyl-2-[6-(1H-pyrazol-4-yl)-1H-indazol-3-yl]-5,7-dihydro-3H-imidazo[4,5-f]indol-6-one was prepared from 4-iodo-1-(2-trimethylsilanyl-ethoxymethyl)-1H-pyrazole and 5-ethyl-7,7-dimethyl-2-[6-(4,4,5,5-tetramethyl-[1,3,2]dioxaborolan-2-yl)-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one.

MS: M=412.3 (ESI+)

¹H-NMR (400 MHz, DMSO): ε (ppm)=1.21 (t, 3H), 1.34 (s, 6H), 3.79 (m, 2H), 7.03 and 7.73 (s, 1H, two tautomeric forms), 7.1 (d, 1H), 7.59 (d, 1H), 7.77 (s, 1H), 8.20 (s, 2H), 8.43 (d, 1H), 12.93 (s, 1H), 13.48 (s, 1H)

### Example 38

### 5-Ethyl-7,7-dimethyl-2-(6-phenylethynyl-1H-indazol-3-yl)-5,7-dihydro-3H-imidazo[4,5-f]indol-6-one

### i) 5-Ethyl-7,7-dimethyl-2-[6-phenylethynyl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one

A mixture of 2-[6-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one (150 mg, 0.219 mmol), ethynyl-benzene (33.5 mg, 0.328 mmol), dichlorobis(triphenylphosphine) palladium (II) (8 mg, 0.011 mmol), copper(I) iodide (5 mg, 0.026 mmol) and diethylamine (426 mg, 600 μl, 5.82 mmol) under an argon atmosphere was heated to 60° C. for 6 h. The reaction mixture was allowed to cool to room temperature and was treated with water. The aqueous phase was extracted three times with ethyl acetate. The combined organic phases were dried over MgSO₄ and the solvent was evaporated. The residue was purified by HPL chromatography to yield 5-ethyl-7,7-dimethyl-2-[6-phenylethynyl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (103.5 mg, 0.146 mmol, 67%).

### ii) 5-Ethyl-7,7-dimethyl-2-(6-phenylethynyl-1H-indazol-3-yl)-5,7-dihydro-3H-imidazo[4,5-f]indol-6-one

In an analogous manner as described for example 4 iii) 5-ethyl-7,7-dimethyl-2-(6-phenylethynyl-1H-indazol-3-yl)-5,7-dihydro-3H-imidazo[4,5-f]indol-6-one was prepared from 5-ethyl-7,7-dimethyl-2-[6-phenylethynyl-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one

MS: M=446.14 (ESI+)

¹H-NMR (400 MHz, DMSO): δ (ppm)=13.74 (m, 1H), 13.04 (m, 1H), 8.53 (m, 1H), 7.85 (s, 1H), 7.73 and 7.47 (s, 1H), 7.63 (m, 2H), 7.46 (m, 4H), 7.38 and 7.04 (s, 1H), 3.79 (m, 2H), 1.34 (s, 6H), 1.21 (t, 3H)

### Example 39

### 5-Ethyl-7,7-dimethyl-2-{6-[2-(3-nitro-phenyl)-vinyl]-1H-indazol-3-yl}-5,7-dihydro-3H-imidazo[4,5-f]indol-6-one

### i) 5-Ethyl-7,7-dimethyl-2-[6-[2-(3-nitro-phenyl)-vinyl]-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one

A mixture of 2-[6-bromo-1-(2-trimethylsilanyl-ethoxymethyl)-1H-indazol-3-yl]-5-ethyl-7,7-dimethyl-3-(2-trimethylsilanyl-ethoxymethyl)-5,7-dihydro-3H-imidazo[4,5-f]indol-6-one (50 mg, 0.073 mmol), 1-nitro-3-vinyl-benzene (16.6 mg, 0.111 mmol), palladium (II) acetate (0.5 mg, 0.0022 mmol), tri-o-tolylphosphin (1.5 mg, 0.0049), triethylamine (14.9 mg, 20.5 μl, 0.147 mmol) and DMF (0.5 ml) under an argon atmosphere was heated to 140° C. for 14 h. The reaction mixture was allowed to cool to room temperature and was treated with water. The aqueous phase was extracted three times with ethyl acetate. The combined organic phases were dried over MgSO₄ and the solvent was evaporated. The residue was purified by HPL chromatography to yield 5-ethyl-7,7-dimethyl-2-[6-[2-(3-nitro-phenyl)-vinyl]-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one (21.5 mg, 0.0285 mmol, 39%).

### ii) 5-Ethyl-7,7-dimethyl-2-{6-[2-(3-nitro-phenyl)-vinyl]-1H-indazol-3-yl}-5,7-dihydro-3H-imidazo[4,5-f]indol-6-one

In an analogous manner as described for example 4 iii) 5-ethyl-7,7-dimethyl-2-{6-[2-(3-nitro-phenyl)-vinyl]-1H-indazol-3-yl}-5,7-dihydro-3H-imidazo[4,5-f]indol-6-one was prepared from 5-ethyl-7,7-dimethyl-2-[6-[2-(3-nitro-phenyl)-vinyl]-1-(2-trimethylsilanyl-ethoxymethyl)-1H-indazol-3-yl]-3-(2-trimethylsilanyl-ethoxymethyl)-5,7-dihydro-3H-imidazo[4,5-f]indol-6-one.

MS: M=493.30 (ESI+)

¹H-NMR (400 MHz, DMSO): δ (ppm)=13.67 (m, 1H), 12.99 (m, 1H), 8.55-8.45 (m, 2H), 8.14 (m, 2H), 7.83 (s, 1H), 7.77-7.70 (m, 3H), 7.69 and 7.45 (s, 1H), 7.64-7.56 (d, 1H), 7.39 and 7.03 (s, 1H), 3.79 (m, 2H), 1.34 (m, 6H), 1.22 (m, 3H)

1. A compound according to formula I, 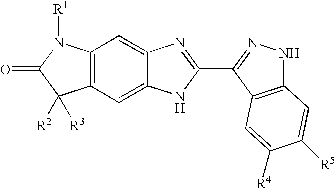
wherein
R¹ is alkyl;
R² and R³ are alkyl;
one of R⁴ and R⁵ is selected from the group consisting of:
a) -X-heteroaryl, wherein the heteroaryl is optionally substituted one to three times by alkyl, alkyl-C(O)—, alkoxy, fluorinated alkyl, fluorinated alkoxy, cyano, nitro, amino, alkylamino, dialkylamino or halogen;
b) -Y-phenyl, wherein the phenyl is:
(i) optionally substituted one to three times by alkyl, alkyl-C(O)—, carboxy, alkyl-NHC(O)—, alkoxy, fluorinated alkyl, fluorinated alkoxy, cyano, hydroxy, nitro, amino, alkylamino, dialkylamino, alkyl-C(O)NH—, alkyl-S(O)₂NH—, halogen, 2,4-dioxa-pentan-1,5-diyl or 2,5-dioxa-hexan-1,6-diyl; or
(ii) substituted once by phenyl; and
c) -Z-cycloalkyl;
and the other of R⁴ and R⁵ is hydrogen;
X is a single bond or —C≡C—;
Y is selected from the group consisting of: a single bond, —CH═CH— and —C≡C—;
Z is —CH═CH—;
or a pharmaceutically acceptable salt thereof.

2. A compound according to claim 1, wherein
one of R⁴ and R⁵ is selected from the group consisting of:
a) -X-heteroaryl, wherein the heteroaryl is optionally substituted one to three times by alkyl or alkoxy;
b) -Y-phenyl, wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl; and
c) -Z-cycloalkyl;
and the other of R⁴ and R⁵ is hydrogen;
X is a single bond;
Y is selected from the group consisting of: a single bond, —CH═CH— and —C≡C—; and
Z is —CH═CH—.

3. A compound according to claim 1, wherein
one of R⁴ and R⁵ is -X-heteroaryl, wherein the heteroaryl is optionally substituted one to three times by alkyl or alkoxy;
and the other of R⁴ and R⁵ is hydrogen.

4. A compound according to claim 1, wherein
one of R⁴ and R⁵ is -Y-phenyl, wherein the phenyl is optionally substituted one to three times by alkyl, alkyl-C(O)—, alkoxy, fluorinated alkyl, nitro, dialkylamino, halogen or 2,4-dioxa-pentan-1,5-diyl; or wherein the phenyl is substituted once by phenyl;
and the other of R⁴ and R⁵ is hydrogen.

5. A compound according to claim 1, wherein
one of R⁴ and R⁵ is -Z-cycloalkyl;
and the other of R⁴ and R⁵ is hydrogen.

6. A compound according claim 1 selected from the group consisting of:
5-Ethyl-7,7-dimethyl-2-[5-(1H-[1,2,4]triazol-3-yl)-1H-indazol-3-yl]-5,7-dihydro-3H-imidazo[4,5-f]indol-6-one;
5-Ethyl-7,7-dimethyl-2-[6-(1H-[1,2,4]triazol-3-yl)-1H-indazol-3-yl]-5,7-dihydro-3H-imidazo[4,5-f]indol-6-one;
5-Ethyl-7,7-dimethyl-2-[5-(1H-tetrazol-5-yl)-1H-indazol-3-yl]-5,7-dihydro-3H-imidazo[4,5-f]indol-6-one;
5-Ethyl-7,7-dimethyl-2-(6-thiophen-3-yl-1H-indazol-3-yl)-5,7-dihydro-3H-imidazo[4,5-f]indol-6-one;
5-Ethyl-7,7-dimethyl-2-[6-(1-methyl-1H-pyrazol-4-yl)-1H-indazol-3-yl]-5,7-dihydro-3H-imidazo[4,5-f]indol-6-one;
5-Ethyl-7,7-dimethyl-2-(6-pyridin-3-yl-1H-indazol-3-yl)-5,7-dihydro-3H-imidazo[4,5-f]indol-6-one;
5-Ethyl-2-[6-(6-methoxy-pyridin-3-yl)-1H-indazol-3-yl]-7,7-dimethyl-5,7-dihydro-3H-imidazo[4,5-f]indol-6-one;
5-Ethyl-7,7-dimethyl-2-(6-pyridin-4-yl-1H-indazol-3-yl)-5,7-dihydro-3H-imidazo[4,5-f]indol-6-one;
5-Ethyl-7,7-dimethyl-2-(6-thiophen-2-yl-1H-indazol-3-yl)-5,7-dihydro-3H-imidazo[4,5-f]indol-6-one;
5-Ethyl-2-[5-(6-methoxy-pyridin-3-yl)-1H-indazol-3-yl]-7,7-dimethyl-5,7-dihydro-3H-imidazo[4,5-f]indol-6-one; compound with acetic acid;
5-Ethyl-7,7-dimethyl-2-(5-thiophen-3-yl-1H-indazol-3-yl)-5,7-dihydro-3H-imidazo[4,5-f]indol-6-one; compound with acetic acid;
5-Ethyl-7,7-dimethyl-2-[5-(1-methyl-1H-pyrazol-4-yl)-1H-indazol-3-yl]-5,7-dihydro-3H-imidazo[4,5-f]indol-6-one; compound with acetic acid;
5-Ethyl-7,7-dimethyl-2-(5-pyridin-3-yl-1H-indazol-3-yl)-5,7-dihydro-3H-imidazo[4,5-f]indol-6-one;
5-Ethyl-7,7-dimethyl-2-(6-pyrimidin-5-yl-1H-indazol-3-yl)-5,7-dihydro-3H-imidazo[4,5-f]indol-6-one;
5-Ethyl-7,7-dimethyl-2-(6-pyridin-2-yl-1H-indazol-3-yl)-5,7-dihydro-3H-imidazo[4,5-f]indol-6-one;
5-Ethyl-7,7-dimethyl-2-(5-pyrimidin-5-yl-1H-indazol-3-yl)-5,7-dihydro-3H-imidazo[4,5-f]indol-6-one;
5-Ethyl-7,7-dimethyl-2-(5-pyridin-2-yl-1H-indazol-3-yl)-5,7-dihydro-3H-imidazo[4,5-f]indol-6-one;
5-Ethyl-7,7-dimethyl-2-[6-(1H-pyrazol-4-yl)-1H-indazol-3-yl]-5,7-dihydro-3H-imidazo[4,5-f]indol-6-one;
2-[6-(4-Dimethylamino-phenyl)-1H-indazol-3-yl]-5-ethyl-7,7-dimethyl-5,7-dihydro-3H-imidazo[4,5-f]indol-6-one;
2-[6-(4-Acetyl-phenyl)-1H-indazol-3-yl]-5-ethyl-7,7-dimethyl-5,7-dihydro-3H-imidazo[4,5-f]indol-6-one;
4-[3-(5-Ethyl-7,7-dimethyl-6-oxo-3,5,6,7-tetrahydro-imidazo[4,5-f]indol-2-yl)-1H-indazol-6-yl]-benzoic acid;
2-(6-Benzo[1,3]dioxol-5-yl-1H-indazol-3-yl)-5-ethyl-7,7-dimethyl-5,7-dihydro-3H-imidazo[4,5-f]indol-6-one;
2-[6-(3-Dimethylamino-phenyl)-1H-indazol-3-yl]-5-ethyl-7,7-dimethyl-5,7-dihydro-3H-imidazo[4,5-f]indol-6-one;
5-Ethyl-7,7-dimethyl-2-[6-(3-nitro-phenyl)-1H-indazol-3-yl]-5,7-dihydro-3H-imidazo[4,5-f]indol-6-one;
2-[5-(4-Dimethylamino-phenyl)-1H-indazol-3-yl]-5-ethyl-7,7-dimethyl-5,7-dihydro-3H-imidazo[4,5-f]indol-6-one;
2-[5-(3-Dimethylamino-phenyl)-1H-indazol-3-yl]-5-ethyl-7,7-dimethyl-5,7-dihydro-3H-imidazo[4,5-f]indol-6-one;
2-(5-Benzo[1,3]dioxol-5-yl-1H-indazol-3-yl)-5-ethyl-7,7-dimethyl-5,7-dihydro-3H-imidazo[4,5-f]indol-6-one; compound with acetic acid;
5-Ethyl-7,7-dimethyl-2-(6-phenyl-1H-indazol-3-yl)-5,7-dihydro-3H-imidazo[4,5-f]indol-6-one;
2-[6-(3,5-Dimethoxy-phenyl)-1H-indazol-3-yl]-5-ethyl-7,7-dimethyl-5,7-dihydro-3H-imidazo[4,5-f]indol-6-one;
5-Ethyl-7,7-dimethyl-2-[6-((E)-styryl)-1H-indazol-3-yl]-5,7-dihydro-3H-imidazo[4,5-f]indol-6-one;
5-Ethyl-2-{6-[(E)-2-(4-fluoro-phenyl)-vinyl]-1H-indazol-3-yl}-7,7-dimethyl-5,7-dihydro-3H-imidazo[4,5-f]indol-6-one;
2-[6-((E)-2-Biphenyl-4-yl-vinyl)-1H-indazol-3-yl]-5-ethyl-7,7-dimethyl-5,7-dihydro-3H-imidazo[4,5-f]indol-6-one;
5-Ethyl-2-{6-[(E)-2-(4-methoxy-phenyl)-vinyl]-1H-indazol-3-yl}-7,7-dimethyl-5,7-dihydro-3H-imidazo[4,5-f]indol-6-one;
5-Ethyl-7,7-dimethyl-2-{6-[(E)-2-(4-trifluoromethyl-phenyl)-vinyl]-1H-indazol-3-yl}-5,7-dihydro-3H-imidazo[4,5-f]indol-6-one;
2-{6-[(E)-2-(4-Chloro-phenyl)-vinyl]-1H-indazol-3-yl}-5-ethyl-7,7-dimethyl-5,7-dihydro-3H-imidazo[4,5-f]indol-6-one;
5-Ethyl-2-{6-[(E)-2-(3-fluoro-phenyl)-vinyl]-1H-indazol-3-yl}-7,7-dimethyl-5,7-dihydro-3H-imidazo[4,5-f]indol-6-one; and
5-Ethyl-7,7-dimethyl-2-{6-[(E)-2-(3-nitro-phenyl)-vinyl]-1H-indazol-3-yl}-5,7-dihydro-3H-imidazo[4,5-f]indol-6-one; compound with acetic acid;
5-Ethyl-7,7-dimethyl-2-(6-phenylethynyl-1H-indazol-3-yl)-5,7-dihydro-3H-imidazo[4,5-f]indol-6-one; and
2-[6-((E)-2-Cyclohexyl-vinyl)-1H-indazol-3-yl]-5-ethyl-7,7-dimethyl-5,7-dihydro-3H-imidazo[4,5-f]indol-6-one.

7. (canceled)

8. A pharmaceutical composition comprising a compound according to claim 1 and a pharmaceutically-acceptable excipient.

9-11. (canceled)
